SEQUENCE LISTING (79 sequences)

SEQ ID NO: 1 - protein, 759 aa, Homo sapiens
GPRAPCAAACTCAGDSLDCGGRGLAALPGDLPSWTRSLNLSYNKLSEIDPAGFEDLPNLQEVYLNNNELTAVPSLGAASSHVVSLFLQHNKIRSVEGSQLKAYLSLEVLDLSLNNITEVRNTCFPHGPPIKELNLAGNRIGTLELGAFDGLSRSLLTLRLSKNRITQLPVRAFKLPRLTQLDLNRNRIRLIEGLTFQGLNSLEVLKLQRNNISKLTDGAFWGLSKMHVLHLEYNSLVEVNSGSLYGLTALHQLHLSNNSIARIHRKGWSFCQKLHELVLSFNNLTRLDEESLAELSSLSVLRLSHNSISHIAEGAFKGLRSLRVLDLDHNEISGTIEDTSGAFSGLDSLSKLTLFGNKIKSVAKRAFSGLEGLEHLNLGGNAIRSVQFDAFVKMKNLKELHISSDSFLCDCQLKWLPPWLIGRMLQAFVTATCAHPESLKGQSIFSVPPESFVCDDFLKPQIITQPETTMAMVGKDIRFTCSAASSSSSPMTFAWKKDNEVLTNADMENFVHVHAQDGEVMEYTTILHLRQVTFGHEGRYQCVITNHFGSTYSHKARLTVNVLPSFTKTPHDITIRTTTVARLECAATGHPNPQIAWQKDGGTDFPAARERRMHVMPDDDVFFITDVKIDDAGVYSCTAQNSAGSISANATLTVLETPSLVVPLEDRVVSVGETVALQCKATGNPPPRITWFKGDRPLSLTERHHLTPDNQLLVVQNVVAEDAGRYTCEMSNTLGTERAHSQLSVLPAAGCRKDGTTVG

SEQ ID NO: 2 - DNA, 2397 bp, Homo sapiens
ggcccgcgggcgccctgcgcggccgcctgcacttgcgctggggactcgctggactgcggtgggcgcgggctggctgcgttgcccggggacctgccctcctggacgcggagcctaaacctgagttacaacaaactctctgagattgaccctgctggttttgaggacttgccgaacctacaggaagtgtacctcaataataatgagttgacagcggtaccatccctgggcgctgcttcatcacatgtcgtctctctctttctgcagcacaacaagattcgcagcgtggaggggagccagctgaaggcctacctttccttagaagtgttagatctgagtttgaacaacatcacggaagtgcggaacacctgctttccacacggaccgcctataaaggagctcaacctggcaggcaatcggattggcaccctggagttgggagcatttgatggtctgtcacggtcgctgctaactcttcgcctgagcaaaaacaggatcacccagcttcctgtaagagcattcaagctacccaggctgacacaactggacctcaatcggaacaggattcggctgatagagggcctcaccttccaggggctcaacagcttggaggtgctgaagcttcagcgaaacaacatcagcaaactgacagatggggccttctggggactgtccaagatgcatgtgctgcacctggagtacaacagcctggtagaagtgaacagcggctcgctctacggcctcacggccctgcatcagctccacctcagcaacaattccatcgctcgcattcaccgcaagggctggagcttctgccagaagctgcatgagttggtcctgtccttcaacaacctgacacggctggacgaggagagcctggccgagctgagcagcctgagtgtcctgcgtctcagccacaattccatcagccacattgcggagggtgccttcaagggactcaggagcctgcgagtcttggatctggaccataacgagatttcgggcacaatagaggacacgagcggcgccttctcagggctcgacagcctcagcaagctgactctgtttggaaacaagatcaagtctgtggctaagagagcattctcggggctggaaggcctggagcacctgaaccttggagggaatgcgatcagatctgtccagtttgatgcctttgtgaagatgaagaatcttaaagagctccatatcagcagcgacagcttcctgtgtgactgccagctgaagtggctgcccccgtggctaattggcaggatgctgcaggcctttgtgacagccacctgtgcccacccagaatcactgaagggtcagagcattttctctgtgccaccagagagtttcgtgtgcgatgacttcctgaagccacagatcatcacccagccagaaaccaccatggctatggtgggcaaggacatccggtttacatgctcagcagccagcagcagcagctcccccatgacctttgcctggaagaaagacaatgaagtcctgaccaatgcagacatggagaactttgtccacgtccacgcgcaggacggggaagtgatggagtacaccaccatcctgcacctccgtcaggtcactttcgggcacgagggccgctaccaatgtgtcatcaccaaccactttggctccacctattcacataaggccaggctcaccgtgaatgtgttgccatcattcaccaaaacgccccacgacataaccatccggaccaccaccgtggcccgcctcgaatgtgctgccacaggtcacccaaaccctcagattgcctggcagaaggatggaggcacggatttccccgctgcccgtgagcgacgcatgcatgtcatgccggatgacgacgtgtttttcatcactgatgtgaaaatagatgacgcaggggtttacagctgtactgctcagaactcagccggttctatttcagctaatgccaccctgactgtcctagagaccccatccttggtggtccccttggaagaccgtgtggtatctgtgggagaaacagtggccctccaatgcaaagccacggggaaccctccgccccgcatcacctggttcaagggggaccgcccgctgagcctcactgagcggcaccacctgacccctgacaaccagctcctggtggttcagaacgtggtggcagaggatgcgggccgatatacctgtgagatgtccaacaccctgggcacggagcgagctcacagccagctgagcgtcctgcccgcagcaggctgcaggaaggatgggaccacggtaggcatcttcaccattgctgtcgtgagcagcatcgtcctgacgtcactggtctgggtgtgcatcatctaccagaccaggaagaagagtgaagagtacagtgtcaccaacacagatgaaaccgtc

SEQ ID NO: 3 - protein, 761 aa, Mus musculus
QAGPRAPCAAACTCAGDSLDCSGRGLATLPRDLPSWTRSLNLSYNRLSEIDSAAFEDLTNLQEVYLNSNELTAIPSLGAASIGVVSLFLQHNKILSVDGSQLKSYLSLEVLDLSSNNITEIRSSCFPNGLRIRELNLASNRISILESGAFDGLSRSLLTLRLSKNRITQLPVKAFKLPRLTQLDLNRNRIRLIEGLTFQGLDSLEVLRLQRNNISRLTDGAFWGLSKMHVLHLEYNSLVEVNSGSLYGLTALHQLHLSNNSISRIQRDGWSFCQKLHELILSFNNLTRLDEESLAELSSLSILRLSHNAISHIAEGAFKGLKSLRVLDLDHNEISGTIEDTSGAFTGLDNLSKLTLFGNKIKSVAKRAFSGLESLEHLNLGENAIRSVQFDAFAKMKNLKELYISSESFLCDCQLKWLPPWLMGRMLQAFVTATCAHPESLKGQSIFSVLPDSFVCDDFPKPQIITQPETTMAVVGKDIRFTCSAASSSSSPMTFAWKKDNEVLANADMENFAHVRAQDGEVMEYTTILHLRHVTFGHEGRYQCIITNHFGSTYSHKARLTVNVLPSFTKIPHDIAIRTGTTARLECAATGHPNPQIAWQKDGGTDFPAARERRMHVMPDDDVFFITDVKIDDMGVYSCTAQNSAGSVSANATLTVLETPSLAVPLEDRVVTVGETVAFQCKATGSPTPRITWLKGGRPLSLTERHHFTPGNQLLVVQNVMIDDAGRYTCEMSNPLGTERAHSQLSILPTPGCRKDGTTVG

SEQ ID NO: 4 - DNA, 2283 bp, Mus musculus
caggctggcccgcgggccccctgcgcggccgcctgcacttgcgccggggactcgctggactgcagtgggcgcgggctggcgacgctgccccgggacctgccctcctggacgcgcagcctaaacctgagttataacagactctccgagatcgactctgctgcttttgaggacttgacgaatctgcaggaagtgtacctcaacagcaatgagctgacagccataccatcactgggcgctgcttccataggagttgtctctctctttttgcagcacaacaagatccttagtgtggatgggagccagctgaagtcgtacctgtccttggaagtgctggatctgagttccaacaacatcacggaaattcggagctcctgtttcccgaacggcctgcgtataagggaactcaacttggcgagcaaccgcatcagcatcctggagtctggagcatttgatggtctgtcgcggtcactgctgactctccgtctgagcaaaaacaggatcacccagcttcctgtgaaagcgttcaagctacccaggctgacacaactagacctgaatcggaatcggattcggctgattgaaggcctcacgttccaggggctcgacagcttagaggtgctgaggcttcagaggaacaacatcagcaggctgacggacggggccttctgggggctgtctaagatgcacgtgctgcacctggagtacaacagtctggtggaagtgaacagtggctccctctatggcctcacagccctgcaccagctgcacctcagcaacaactccatctctcgaattcagcgtgatggctggagcttctgccaaaagctgcatgagttgattctgtccttcaacaacctcacgcggctggatgaggagagtctagcggagttgagcagcctcagtatcctgcgcctcagtcacaacgccatcagtcacattgctgaaggcgccttcaagggactcaagagtctgcgggtcttggacctggaccataacgagatctcgggtacaatcgaggataccagtggtgcctttacggggcttgacaacctcagcaagctgactctgtttggaaacaagatcaaatctgtggctaagagagccttctcgggcctggaaagcctggaacacctgaaccttggagagaatgcaatcaggtctgtccagtttgatgcctttgcaaagatgaagaaccttaaagagctctacatcagcagtgagagcttcctgtgtgactgccagctcaagtggctgcccccatggctaatgggtaggatgctgcaggcctttgtgacagccacctgtgcccatccagagtcgctgaagggccagagcattttctcagtgctgccagacagctttgtgtgtgatgactttccaaagccacagatcatcacccagcctgagacgaccatggctgtggtgggcaaggacatccgtttcacatgctccgcagccagcagcagcagctcaccaatgaccttcgcctggaagaaggacaatgaggtcctggccaatgcagacatggagaactttgcccacgtccgtgcacaggacggcgaagtgatggagtataccactatcctgcacctccgtcacgtcacctttgggcacgagggccgctaccagtgtatcatcacaaaccactttggctccacatactcccacaaagccaggctcactgtgaatgtgttgccatcattcactaaaataccccatgacattgccatccggactggcaccacagcccgcctcgagtgtgctgccacgggccaccctaaccctcagattgcctggcagaaggatggaggcaccgatttcccggcagctcgtgagcgacgcatgcatgttatgccagacgatgatgtgttcttcatcactgatgtgaaaatagacgacatgggggtctacagctgcactgcccagaactcggcaggctcggtttcagccaacgctaccctcacagtcttagaaactccatccttggcagtgcctctggaagaccgtgtggtaactgtgggagaaacagtggccttccagtgcaaagcaaccgggagccccacaccacgcatcacctggcttaagggaggtcgcccattgagcctcacagagcgccaccatttcactccaggcaaccagctgctggttgttcagaatgtgatgatagacgatgcagggcggtatacctgtgagatgtctaatcccctgggcactgagcgagcacatagccagctgagcattttacctacccctggctgccggaaggatgggaccaccgtaggc

SEQ ID NO: 5 - protein, 8 aa, Artificial Sequence
  GTC210-01 heavy chain CDR1
GFTFSGYD

SEQ ID NO: 6 - protein, 9 aa, Artificial Sequence
  GTC210-01 heavy chain CDR2
LIYPDSGNK

SEQ ID NO: 7 - protein, 14 aa, Artificial Sequence
  GTC210-01 heavy chain CDR3
ARDAGLSWAGAFDY

SEQ ID NO: 8 - protein, 8 aa, Artificial Sequence
  GTC210-01 light chain CDR1
SSNIGSNY

SEQ ID NO: 9 - protein, 3 aa, Artificial Sequence
  GTC210-01 light chain CDR2
SDS

SEQ ID NO: 10 - protein, 11 aa, Artificial Sequence
  GTC210-01 light chain CDR3
GSWDYSLSAYV

SEQ ID NO: 11 - protein, 121 aa, Artificial Sequence
  GTC210-01 heavy chain_variable region
EVQLLESGGGLVQPGGSLRLSCAASGFTFSGYDMSWVRQAPGKGLEWVSLIYPDSGNKYYADSVKGRFTISRDNSKNTLYLQMNSLRAEDTAVYYCARDAGLSWAGAFDYWGQGTLVTVSS

SEQ ID NO: 12 - protein, 110 aa, Artificial Sequence
  GTC210-01 light chain_variable region
QSVLTQPPSASGTPGQRVTISCSGSSSNIGSNYVTWYQQLPGTAPKLLIYSDSHRPSGVPDRFSGSKSGTSASLAISGLQSEDEADYYCGSWDYSLSAYVFGGGTKLTVL

SEQ ID NO: 13 - protein, 8 aa, Artificial Sequence
  GTC110-04 heavy chain CDR1
GFTFSNYA

SEQ ID NO: 14 - protein, 9 aa, Artificial Sequence
  GTC110-04 heavy chain CDR2
VISHGGGST

SEQ ID NO: 15 - protein, 20 aa, Artificial Sequence
  GTC110-04 heavy chain CDR3
ARVISNCHLGVCYYSNGMDV

SEQ ID NO: 16 - protein, 8 aa, Artificial Sequence
  GTC110-04 light chain CDR1
SSNIGNND

SEQ ID NO: 17 - protein, 3 aa, Artificial Sequence
  GTC110-04 light chain CDR2
SDS

SEQ ID NO: 18 - protein, 11 aa, Artificial Sequence
  GTC110-04 light chain CDR3
GTWDYSLSGYV

SEQ ID NO: 19 - protein, 127 aa, Artificial Sequence
  GTC110-04 heavy chain_variable region
EVQLLESGGGLVQPGGSLRLSCAASGFTFSNYAMSWVRQAPGKGLEWVSVISHGGGSTYYADSVKGRFTISRDNSKNTLYLQMNSLRAEDTAVYYCARVISNCHLGVCYYSNGMDVWGQGTLVTVSS

SEQ ID NO: 20 - protein, 110 aa, Artificial Sequence
  GTC110-04 light chain_variable region
QSVLTQPPSASGTPGQRVTISCSGSSSNIGNNDVYWYQQLPGTAPKLLIYSDSQRPSGVPDRFSGSKSGTSASLAISGLRSEDEADYYCGTWDYSLSGYVFGGGTKLTVL

SEQ ID NO: 21 - protein, 328 aa, Mus musculus
TTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 22 - protein, 107 aa, Mus musculus
RTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 23 - protein, 330 aa, Homo sapiens [sequence omitted]

SEQ ID NO: 24 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 107 aa, Homo sapiens [sequence omitted]

SEQ ID NO: 26 - protein, 326 aa, Homo sapiens
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPGK

SEQ ID NO: 27 - protein, 377 aa, Homo sapiens
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRVELKTPLGDTTHTCPRCPEPKSCDTPPPCPRCPEPKSCDTPPPCPRCPEPKSCDTPPPCPRCPAPELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTKGQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 28 - protein, 327 aa, Homo sapiens
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 29 - protein, 245 aa, Artificial Sequence
  Hybrid Fc_Heavy region
RNTGRGGEEKKKEKEKEEQEERETKTPECPSHTQPLGVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - protein, 451 aa, Artificial Sequence
  GTC210-01 heavy chain_mouse IgG2 Fc_full sequence
EVQLLESGGGLVQPGGSLRLSCAASGFTFSGYDMSWVRQAPGKGLEWVSLIYPDSGNKYYADSVKGRFTISRDNSKNTLYLQMNSLRAEDTAVYYCARDAGLSWAGAFDYWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 31 - protein, 217 aa, Artificial Sequence
  GTC210-01 light chain_mouse IgG2 Fc_full sequence
QSVLTQPPSASGTPGQRVTISCSGSSSNIGSNYVTWYQQLPGTAPKLLIYSDSHRPSGVPDRFSGSKSGTSASLAISGLQSEDEADYYCGSWDYSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 32 - protein, 457 aa, Artificial Sequence
  GTC110-04 heavy chain_mouse IgG2 Fc_full sequence
EVQLLESGGGLVQPGGSLRLSCAASGFTFSNYAMSWVRQAPGKGLEWVSVISHGGGSTYYADSVKGRFTISRDNSKNTLYLQMNSLRAEDTAVYYCARVISNCHLGVCYYSNGMDVWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 33 - protein, 217 aa, Artificial Sequence
  GTC110-04 light chain_mouse IgG2 Fc_full sequence
QSVLTQPPSASGTPGQRVTISCSGSSSNIGNNDVYWYQQLPGTAPKLLIYSDSQRPSGVPDRFSGSKSGTSASLAISGLRSEDEADYYCGTWDYSLSGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 34 - protein, 458 aa, Artificial Sequence
  GTC110-04 heavy chain_human IgG1
EVQLLESGGGLVQPGGSLRLSCAASGFTFSNYAMSWVRQAPGKGLEWVSVISHGGGSTYYADSVKGRFTISRDNSKNTLYLQMNSLRAEDTAVYYCARVISNCHLGVCYYSNGMDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGKY

SEQ ID NO: 35 - protein, 217 aa, Artificial Sequence
  GTC110-04 light chain_human IgG1
QSVLTQPPSASGTPGQRVTISCSGSSSNIGNNDVYWYQQLPGTAPKLLIYSDSQRPSGVPDRFSGSKSGTSASLAISGLRSEDEADYYCGTWDYSLSGYVFGGGTKLTVLRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 36 - protein, 48 aa, Homo sapiens
GDSLDCGGRGLAALPGDLPSSTRSLNLSYNKLSEIDPAGFEDLPNLQE

SEQ ID NO: 37 - protein, 57 aa, Homo sapiens
SDSFLCDCQLKWLPPWLIGRMLQAFVTATCAHPESLKGQSIFSVPPESFVCDDFLKA

SEQ ID NO: 38 - protein, 7 aa, Homo sapiens
NNNELTA

SEQ ID NO: 39 - protein, 18 aa, Homo sapiens
NAIRSVQFDAFVKMKNLK

SEQ ID NO: 40 - protein, 5 aa, Homo sapiens
NKIRS

SEQ ID NO: 41 - protein, 6 aa, Homo sapiens
VEVNSG

SEQ ID NO: 42 - protein, 6 aa, Homo sapiens
ARIHRK

SEQ ID NO: 43 - protein, 9 aa, Homo sapiens
NNITEVRNT

SEQ ID NO: 44 - protein, 14 aa, Homo sapiens
IRLIEGLTFQGLNS

SEQ ID NO: 45 - protein, 14 aa, Homo sapiens
ISKLTDGAFWGLSK

SEQ ID NO: 46 - protein, 13 aa, Homo sapiens
NRITQLPVRAFKL

SEQ ID NO: 47 - protein, 6 aa, Homo sapiens
KAYLSL

SEQ ID NO: 48 - protein, 25 aa, Homo sapiens
KKDNEVLTNADMENFVHVHAVMEYT

SEQ ID NO: 49 - protein, 11 aa, Homo sapiens
SAASSSSSPMT

SEQ ID NO: 50 - protein, 18 aa, Homo sapiens
FKGDRPLSLTERHHLTPD

SEQ ID NO: 51 - protein, 10 aa, Homo sapiens
QLSVLLENLY

SEQ ID NO: 52 - protein, 8 aa, Homo sapiens
KPQIITQP

SEQ ID NO: 53 - protein, 15 aa, Homo sapiens
VPLEDRVVSVGETVA

SEQ ID NO: 54 - protein, 10 aa, Habromys lophurus
HVMPDDDVFF

SEQ ID NO: 55 - protein, 12 aa, Homo sapiens
ITNHFGSTYSHK

SEQ ID NO: 56 - protein, 15 aa, Homo sapiens
QLLVVQNVVAEDAGR

SEQ ID NO: 57 - protein, 8 aa, Homo sapiens
HLRQVTFG

SEQ ID NO: 58 - protein, 451 aa, Artificial Sequence
  GTC210-02 Heavy chain
EVQLLESGGGLVQPGGSLRLSCAASGFTFSNYYMSWVRQAPGKGLEWVSGISPGDSSTYYADSVKGRFTISRDNSKNTLYLQMNSLRAEDTAVYYCAKGLYSNPNEPFDYWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 59 - protein, 217 aa, Artificial Sequence
  GTC210-02 Light chain
QSVLTQPPSASGTPGQRVTISCTGSSSNIGSNYVSWYQQLPGTAPKLLIYDDSQRPSGVPDRFSGSKSGTSASLAISGLRSEDEADYYCGTWDYSLNGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 60 - protein, 457 aa, Artificial Sequence
  GTC210-03 Heavy chain
EVQLLESGGGLVQPGGSLRLSCAASGFTFSSYDMSWVRQAPGKGLEWVSGISPDGSNIYYADSVKGRFTISRDNSKNTLYLQMNSLRAEDTAVYYCAKVGLRCRYEACSYAYGMDVWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 61 - protein, 217 aa, Artificial Sequence
  GTC210-03 Light chain
QSVLTQPPSASGTPGQRVTISCSGSSSNIGSNYVSWYQQLPGTAPKLLIYSDSHRPSGVPDRFSGSKSGTSASLAISGLRSEDEADYYCATWDSSLNGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 62 - protein, 451 aa, Artificial Sequence
  GTC210-04 Heavy chain
EVQLLESGGGLVQPGGSLRLSCAASGFTFSNYDMSWVRQAPGKGLEWVSSISPSSGSIYYADSVKGRFTISRDNSKNTLYLQMNSLRAEDTAVYYCAKDLDAFWRPSFDYWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 63 - protein, 217 aa, Artificial Sequence
  GTC210-04 Light chain
QSVLTQPPSASGTPGQRVTISCTGSSSNIGNNNVNWYQQLPGTAPKLLIYSDSHRPSGVPDRFSGSKSGTSASLAISGLRSEDEADYYCGSWDDSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 64 - protein, 457 aa, Artificial Sequence
  GTC110-01 Heavy chain
EVQLLESGGGLVQPGGSLRLSCAASGFTFSDYDMSWVRQVPGKGLEWVSWISHGGGSIYYADSVKGRFTISRDNSKNTLYLQMNSLRAEDTAVYYCARGLGLCKTGLCYYYDAMDVWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 65 - protein, 217 aa, Artificial Sequence
  GTC110-01 Light chain
QSVLTQPPSASGTPGQRVTISCTGSSSNIGNNSVTWYQQLPGTAPKLLIYADNNRPSGVPDRFSGSKSGTSASLAISGLRSEDEADYYCAAWDSSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 66 - protein, 446 aa, Artificial Sequence
  GTC110-02 Heavy chain
EVQLLESGGGLVQPGGSLRLSCAASGFTFSDYYMSWVRQAPGKGLEWVSGISHDSGSKYYADSVKGRFTISRDNSKNTLYLQMNSLRAEDTAVYYCARHWTTFDYWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 67 - protein, 217 aa, Artificial Sequence
  GTC110-02 Light chain
QSVLTQPPSASGTPGQRVTISCSGSSSNIGSNNVTWYQQLPGTAPKLLIYANSNRPSGVPDRFSGSKSGTSASLAISGLRSEDEADYYCGAWDYSLSAYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 68 - protein, 457 aa, Artificial Sequence
  GTC110-03 Heavy chain
EVQLLESGGGLVQPGGSLRLSCAASGFTFSNYAMSWVRQAPGKGLEWVSAIYPGGGSIYYADSVKGRFTISRDNSKNTLYLQMNSLRAEDTAVYYCARDILPCPWGRCYYDYAMDVWGQGTLVTVSSASTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEPRGPTIKPCPPCKCPAPNLLGGPSVFIFPPKIKDVLMISLSPIVTCVVVDVSEDDPDVQISWFVNNVEVHTAQTQTHREDYNSTLRVVSALPIQHQDWMSGKEFKCKVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 69 - protein, 217 aa, Artificial Sequence
  GTC110-03 Light chain
QSVLTQPPSASGTPGQRVTISCSDSSSNIGSNTVSWYQQLPGTAPKLLIYADNNRPSGVPDRFSGSKSGTSASLAISGLRSEDEADYYCGTWDYSLSGYVFGGGTKLTVLRTVAAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 70 - DNA, 24 bp, Artificial Sequence
  Mouse Lrig-1 forward primer
gacggaattcagtgaggagaacct

SEQ ID NO: 71 - DNA, 24 bp, Artificial Sequence
  Mouse Lrig-1 reverse primer
caactggtagtggcagcttgtagg

SEQ ID NO: 72 - DNA, 24 bp, Artificial Sequence
  Mouse Lrig-2 forward primer
tcacaaggaacattgtctgaacca

SEQ ID NO: 73 - DNA, 24 bp, Artificial Sequence
  Mouse Lrig-2 reverse primer
gcctgatctaacacatcctcctca

SEQ ID NO: 74 - DNA, 24 bp, Artificial Sequence
  Mouse Lrig-3 forward primer
cagcaccttgagctgaacagaaac

SEQ ID NO: 75 - DNA, 24 bp, Artificial Sequence
  Mouse Lrig-3 reverse primer
ccagcctttggtaatctcggttag

SEQ ID NO: 76 - DNA, 24 bp, Artificial Sequence
  Mouse FOXP3 forward primer
ctttcacctatcccacccttatcc

SEQ ID NO: 77 - DNA, 24 bp, Artificial Sequence
  Mouse FOXP3 reverse primer
attcatctacggtccacactgctc

SEQ ID NO: 78 - DNA, 20 bp, Artificial Sequence
  ACTG1 forward primer
ggcgtcatggtgggcatggg

SEQ ID NO: 79 - DNA, 20 bp, Artificial Sequence
  ACTG1 reverse primer
atggcgtggggaagggcgta

(19) United States
(12) Patent Application Publication
(10) Pub. No.: US 2022/0213186 A1
(43) Pub. Date: Jul. 7, 2022

(54) COMPOSITION FOR PREVENTING OR TREATING BRAIN AND NERVOUS SYSTEM DISEASE

(71) Applicant: GOOD T CELLS, INC., Seoul (KR)

(72) Inventors: Jung Ho Kim, Seoul (KR); Beom Seok Kim, Seoul (KR)

(73) Assignee: GOOD T CELLS, INC., Seoul (KR)

(21) Appl. No.: 17/440,733

(22) PCT Filed: Mar. 20, 2020
(86) PCT No.: PCT/KR2020/003903

§ 371 Date: Sep. 17, 2021

(30) Foreign Application Priority Data
Mar. 20, 2019 (KR) 10-2019-0031630

(51) Int. Cl. C07K 16/28; A61P 25/28

(52) U.S. Cl. CPC C07K 16/28; A61P 25/28; C07K 2317/515; C07K 2317/52; C07K 2317/51; C07K 2317/565

(57) ABSTRACT

The present invention relates to a composition for preventing, ameliorating, or treating a brain and nervous system disease by using a binding molecule capable of specifically binding to Lrig-1 protein which is a protein present on the surface of regulatory T cells.

### CROSS-REFERENCE TO RELATED APPLICATIONS

This application is U.S. national phase application under 35 U.S.C. § 371 of International Application No. PCT/KR2020/003903, filed on Mar. 20, 2020, which claims the benefit of priority to Korean Patent Application No. KR10-2019-0031630, filed on Mar. 20, 2019, and the contents of each of which are incorporated herein by reference in their entirety.

### SEQUENCE LISTING

The ASCII file, entitled 21-2031-WO-US_Substitute Sequence Listing.txt, created on Sep. 17, 2021, comprising 100,000 bytes, submitted concurrently with the filing of this application is incorporated herein by reference. The substitute Sequence Listing contains the sequence information forming part of the parent application.

### TECHNICAL FIELD

The present invention relates to a composition for preventing or treating a brain and nervous system disease.

### BACKGROUND ART

In degenerative brain and nervous system diseases such as stroke, dementia, and Alzheimer's disease, deterioration in memory, attention, cognitive ability, emotion regulation, and the like is observed, which results from death of neuronal cells and atrophy of nerve branches. Nerve branch elongation in neuronal cells leads to increased neuroplasticity, and thus plays an important role in memory and learning functions of neural circuitry. Therefore, it is predicted that active ingredients, which promote nerve branch elongation and nerve regeneration in neuronal cells, have the potential to be developed as new therapeutic agents for degenerative brain and nervous system diseases.

As the aging population rapidly increases, the incidence of degenerative brain and nervous system diseases is also on the rise. Despite innovative advances in medicine, prophylactic and therapeutic methods for degenerative brain and nervous system diseases are not yet clearly established, and no drugs have been found which have a decisive effect. Currently, therapeutic agents and treatment methods for degenerative brain and nervous system diseases are being developed; however, they often exhibit side effects and toxicity due to long-term use, and only have an effect of alleviating symptoms rather than treating the disease. Therefore, there is an urgent need to develop a material capable of achieving treatment while having decreased side effects and toxicity.

### Technical Problem

An object of the present invention is to provide a composition for various uses which is capable of preventing, ameliorating, or treating a brain and nervous system disease, in particular, a neurodegenerative disease or neuroinflammatory disease.

However, the technical problem to be achieved by the present invention is not limited to the above-mentioned problems, and other problems that are not mentioned will be clearly understood by those of ordinary skill in the art from the following description.

### Solution to Problem

According to an embodiment of the present invention, there is provided a composition for preventing, ameliorating, or treating a brain and nervous system disease, comprising, as an active ingredient, a binding molecule that specifically binds to leucine-rich and immunoglobulin-like domains 1 (Lrig-1) protein present on the surface of regulatory T cells (Treg cells).

As used herein, the term “binding molecule” refers to a variable domain comprising an intact immunoglobulin that includes a monoclonal antibody, such as a chimeric, humanized, or human monoclonal antibody, or an immunoglobulin that binds to an antigen, for example, an immunoglobulin fragment that competes with intact immunoglobulins for binding to monomeric HA or trimeric HA of influenza A virus. Regardless of the structure, an antigen-binding fragment binds to the same antigen recognized by intact immunoglobulins. The antigen-binding fragment may include a peptide or polypeptide which contains, out of the amino acid sequence of the binding molecule, an amino acid sequence of two or more contiguous residues, 20 or more contiguous amino acid residues, 25 or more contiguous amino acid residues, 30 or more contiguous amino acid residues, 35 or more contiguous amino acid residues, 40 or more contiguous amino acid residues, 50 or more contiguous amino acid residues, 60 or more contiguous amino acid residues, 70 or more contiguous amino acid residues, 80 or more contiguous amino acid residues, 90 or more contiguous amino acid residues, 100 or more contiguous amino acid residues, 125 or more contiguous amino acid residues, 150 or more contiguous amino acid residues, 175 or more contiguous amino acid residues, 200 or more contiguous amino acid residues, or 250 or more contiguous amino acid residues. The term “antigen-binding fragment”, in particular, includes Fab, F(ab′), F(ab′)2, Fv, dAb, Fd, complementarity determining region (CDR) fragments, single-chain antibodies (scFvs), bivalent single-chain antibodies, single-chain phage antibodies, diabodies, triabodies, tetrabodies, polypeptides containing one or more fragments of immunoglobulin which is sufficient for a particular antigen to bind to the polypeptide, and the like. The fragment may be produced synthetically or by enzymatic or chemical digestion of a complete immunoglobulin, or may be produced by genetic engineering methods using recombinant DNA techniques. Production methods are well known in the art.

In the present invention, the “Lrig-1 protein” is a transmembrane protein consisting of 1091 amino acids present on the surface of regulatory T cells, and is composed of leucine-rich repeats (LRRs) and three immunoglobulin-like domains on the extracellular or lumen side, a cell transmembrane sequence, and a cytoplasmic tail portion. The LRIG gene family includes LRIG1, LRIG2, and LRIG3, and the amino acids therebetween are highly conserved. The LRIG1 gene is highly expressed in normal skin and can be expressed in basal and hair follicle cells to regulate proliferation of epithelial stem cells. Therefore, the LRIG1 gene plays an important role in maintaining homeostasis of the epidermis, and its absence may develop psoriasis or skin cancer. It has been reported that in a case where chromosome 3p14.3 portion in which LRIG1 is located is cut off, there is a possibility of developing into cancer cells. In fact, it was identified that expression of LRIG1 is greatly decreased in renal cell carcinoma and cutaneous squamous cell carcinoma. Recently, it has been also found that Lrig-1 is expressed in only about 20 to 30% of cancers. On the other hand, for the purpose of the present invention, the Lrig-1 protein may be, but is not limited to, a protein present in humans or mice.

In the present invention, the Lrig-1 protein may be, but is not limited to, a human-derived polypeptide represented by SEQ ID NO: 1 or a mouse-derived polypeptide represented by SEQ ID NO: 3.

In addition, in the present invention, the Lrig-1 protein represented by SEQ ID NO: 1 may be encoded by a polynucleotide represented by SEQ ID NO: 2, but is not limited thereto.

In addition, in the present invention, the Lrig-1 protein represented by SEQ ID NO: 3 may be encoded by a polynucleotide represented by SEQ ID NO: 4, but is not limited thereto.

In the present invention, the binding molecule may be a binding molecule, comprising:

a heavy chain variable region that contains a heavy chain CDR1 consisting of the amino acid sequence represented by SEQ ID NO: 5 or 13, a heavy chain CDR2 consisting of the amino acid sequence represented by SEQ ID NO: 6 or 14, and a heavy chain CDR3 consisting of the amino acid sequence represented by SEQ ID NO: 7 or 15; and

a light chain variable region that contains a light chain CDR1 consisting of the amino acid sequence represented by SEQ ID NO: 8 or 16, a light chain CDR2 consisting of the amino acid sequence represented by SEQ ID NO: 9 or 17, and a light chain CDR3 consisting of the amino acid sequence represented by SEQ ID NO: 10 or 18.

In the present invention, the binding molecule may be a binding molecule, comprising:

a heavy chain variable region selected from the group consisting of (a) a heavy chain variable region that contains a heavy chain CDR1 represented by SEQ ID NO: 5, a heavy chain CDR2 represented by SEQ ID NO: 6, and a heavy chain CDR3 represented by SEQ ID NO: 7; and

(b) a heavy chain variable region that contains a heavy chain CDR1 represented by SEQ ID NO: 13, a heavy chain CDR2 represented by SEQ ID NO: 14, and a heavy chain CDR3 represented by SEQ ID NO: 15; and

a light chain variable region selected from the group consisting of (c) a light chain variable region that contains a light chain CDR1 represented by SEQ ID NO: 8, a light chain CDR2 represented by SEQ ID NO: 9, and a light chain CDR3 represented by SEQ ID NO: 10; and

(d) a light chain variable region that contains a light chain CDR1 represented by SEQ ID NO: 16, a light chain CDR2 represented by SEQ ID NO: 17, and a light chain CDR3 represented by SEQ ID NO: 18.

In the present invention, the binding molecule may be a binding molecule selected from the group consisting of the following (1) and (2):

(1) a binding molecule comprising a heavy chain variable region that contains a heavy chain CDR1 represented by SEQ ID NO: 5, a heavy chain CDR2 represented by SEQ ID NO: 6, and a heavy chain CDR3 represented by SEQ ID NO: 7; and a light chain variable region that contains a light chain CDR1 represented by SEQ ID NO: 8, a light chain CDR2 represented by SEQ ID NO: 9, and a light chain CDR3 represented by SEQ ID NO: 10; and

(2) a binding molecule comprising a heavy chain variable region that contains a heavy chain CDR1 represented by SEQ ID NO: 13, a heavy chain CDR2 represented by SEQ ID NO: 14, and a heavy chain CDR3 represented by SEQ ID NO: 15; and a light chain variable region that contains a light chain CDR1 represented by SEQ ID NO: 16, a light chain CDR2 represented by SEQ ID NO: 17, and a light chain CDR3 represented by SEQ ID NO: 18.

In the present invention, the binding molecule may be a binding molecule, comprising:

a heavy chain variable region consisting of the amino acid sequence represented by SEQ ID NO: 11 or 19; and

a light chain variable region consisting of the amino acid sequence represented by SEQ ID NO: 12 or 20.

In the present invention, the binding molecule may be a binding molecule selected from the group consisting of the following binding molecules:

a binding molecule comprising a heavy chain variable region represented by SEQ ID NO: 11, and a light chain variable region represented by SEQ ID NO: 12; and

a binding molecule comprising a heavy chain variable region represented by SEQ ID NO: 19, and a light chain variable region represented by SEQ ID NO: 20.

In the present invention, the binding molecule may further comprise a fragment crystallization (Fc) region or a constant region. Here, the Fc region may be an Fc region of an IgA, IgD, IgE, IgM, IgG1, IgG2, IgG3, or IgG4 antibody, or may be derived therefrom. Alternatively, the Fc region may be a hybrid Fc region.

In the present invention, the Fc region may be an Fc region of a mammalian-derived IgA, IgD, IgE, IgM, IgG1, IgG2, IgG3, or IgG4 antibody, and may preferably be an Fc region of a human-derived IgA, IgD, IgE, IgM, IgG1, IgG2, IgG3, or IgG4 antibody. However, the Fc region is not limited thereto.

As an example of the present invention, the constant region may be a mouse-derived IgG2a constant region represented by SEQ ID NO: 21, but is not limited thereto.

As an example of the present invention, the constant region may be a mouse-derived immunoglobulin kappa constant region represented by SEQ ID NO: 22, but is not limited thereto.

As an example of the present invention, the constant region may be a human-derived IgG1 constant region represented by SEQ ID NO: 23 or 24, but is not limited thereto.

As an example of the present invention, the constant region may be a human-derived immunoglobulin kappa constant region represented by SEQ ID NO: 25, but is not limited thereto.

As an example of the present invention, the constant region may be a human-derived IgG2 constant region represented by SEQ ID NO: 26, but is not limited thereto.

As an example of the present invention, the constant region may be a human-derived IgG3 constant region represented by SEQ ID NO: 27, but is not limited thereto.

As an example of the present invention, the constant region may be a human-derived IgG4 constant region represented by SEQ ID NO: 28, but is not limited thereto.

As an example of the present invention, the Fc region may be a human-derived immunoglobulin lambda constant region, but is not limited thereto.

In the present invention, the “hybrid Fc” may be derived from a combination of human IgG subclasses or a combination of human IgD and IgG. In a case where the hybrid Fc binds to a biologically active molecule, polypeptide, or the like, the hybrid Fc has effects of not only increasing a serum half-life of the biologically active molecule, but also increasing an expression level of the polypeptide when a nucleotide sequence encoding the Fc-polypeptide fusion protein is expressed.

As an example of the present invention, the hybrid Fc region may be hybrid Fc represented by SEQ ID NO: 29, but is not limited thereto.

In the binding molecule of the present invention, the Fc or constant region may be linked, via a linker, to the variable region. Here, the linker may be linked to the C-terminus of the Fc or constant region, and the N-terminus of the binding molecule of the present invention may be linked to the linker. However, the present invention is not limited thereto.

In the present invention, the “linker” may contain a sequence that can be cleaved by an enzyme that is overexpressed in a tissue or cell having a target disease. In a case where the linker may be cleaved by the overexpressed enzyme as described above, it is possible to effectively prevent activity of a polypeptide from decreasing due to the Fc or constant region. In the present invention, an example of the linker may be preferably a peptide linker consisting of 33 amino acids located in the 282^{nd} to 314^{th} portion of human albumin which is most abundantly present in the blood, and more preferably a peptide linker consisting of 13 amino acids located in the 292^{nd} to 304^{th} portion of the human albumin. Such portions are portions which are mostly exposed to the outside in three-dimensional structure, and thus have a minimum possibility of inducing an immune response in the body. However, the linker is not limited thereto.

The binding molecule of the present invention may further comprise a heavy chain constant region consisting of an amino acid sequence selected from the group consisting of SEQ ID NOs: 21, 23, 24, 26, 27, 28, and 29.

The binding molecule of the present invention may further comprise a light chain constant region consisting of the amino acid sequence represented by SEQ ID NO: 22 or 25.

The binding molecule of the present invention may further comprise:

a heavy chain constant region consisting of the amino acid sequence represented by SEQ ID NO: 21; and

a light chain constant region consisting of the amino acid sequence represented by SEQ ID NO: 22.

The binding molecule of the present invention may further comprise:

a heavy chain constant region consisting of the amino acid sequence represented by SEQ ID NO: 23, 24, 26, 27, or 28; and

a light chain constant region consisting of the amino acid sequence represented by SEQ ID NO: 25.

The binding molecule of the present invention may further comprise:

a heavy chain constant region consisting of the amino acid sequence represented by SEQ ID NO: 29.

The binding molecule of the present invention may be a binding molecule selected from the group of the following binding molecules:

a binding molecule comprising a heavy chain represented by SEQ ID NO: 30, and a light chain represented by SEQ ID NO: 31;

a binding molecule comprising a heavy chain represented by SEQ ID NO: 32, and a light chain represented by SEQ ID NO: 33; and

a binding molecule comprising a heavy chain represented by SEQ ID NO: 34, and a light chain represented by SEQ ID NO: 35.

The binding molecule of the present invention is characterized by being an antibody, but is not limited thereto. The antibody includes all of a monoclonal antibody, a full-length antibody, or an antibody fragment which is a portion of an antibody, has the ability to bind to Lrig-1 protein, and can compete with the binding molecule of the present invention in binding to an epitope on Lrig-1.

As used herein, the term “antibody” refers to a protein molecule which serves as a receptor that specifically recognizes an antigen, including an immunoglobulin molecule that is immunologically reactive with a particular antigen. For the purpose of the present invention, the antigen may be Lrig-1 protein present on the surface of regulatory T cells. Preferably, the antibody may specifically recognize the leucine-rich region or immunoglobulin-like domain of the Lrig-1 protein, but is not limited thereto.

In the present invention, the “immunoglobulin” has a heavy chain and a light chain, and each of the heavy chain and the light chain comprises a constant region and a variable region. The variable region of each of the light chain and the heavy chain contains three hypervariable regions called complementarity determining regions (hereinafter referred to as “CDRs”) and four framework regions. The CDRs primarily serve to bind to an epitope on an antigen. The CDRs of each chain are typically referred to as CDR1, CDR2, and CDR3 sequentially starting from the N-terminus, and are also distinguished by the chain where particular CDRs are located.

In addition, as used herein, the term “monoclonal antibody” refers to an antibody molecule of a single molecular composition which is obtained from substantially the same antibody population, and exhibits single binding specificity and affinity for a particular epitope.

In the present invention, the “full-length antibody” has a structure with two full-length light chains and two full-length heavy chains in which each light chain is linked to a heavy chain by a disulfide bond, and includes IgA, IgD, IgE, IgM, and IgG. The IgG includes, as subtypes thereof, IgG1, IgG2, IgG3, and IgG4.

In addition, as used herein, the term “antigen fragment ” refers to a fragment that retains an antigen-binding function, and includes Fab, Fab′, F(ab′)₂, Fv, and the like. The Fab has a structure with variable regions of light and heavy chains, a constant region of the light chain, and a first constant region (CH1 domain) of the heavy chain, and has one antigen-binding site. In addition, Fab′ is different from Fab in that Fab′ has a hinge region containing at least one cysteine residue at the C-terminus of the heavy chain CH1 domain. F(ab′)2 antibodies are produced with cysteine residues at the hinge region of Fab′ forming a disulfide bond. Fv (variable fragment) refers to the smallest antibody fragment having only a heavy chain variable region and a light chain variable region. Double-chain Fv (dsFv) is configured to be such that a heavy chain variable region and a light chain variable region are linked to each other by a disulfide bond, and single-chain Fv (scFv) is configured to be such that a heavy chain variable region and a light chain variable region are covalently linked to each other, in general, via a peptide linker. The antibody fragment may be obtained as Fab or F(ab′)₂ fragment in a case where a proteolytic enzyme, for example, papain or pepsin is used, and may be produced through a genetic recombinant technique.

In addition, in the present invention, the antibody may be, but is not limited to, a chimeric antibody, a humanized antibody, a bivalent, bispecific molecule, a minibody, a domain antibody, a bispecific antibody, an antibody mimetic, a unibody, a diabody, a triabody, or a tetrabody, or a fragment thereof.

In the present invention, the “chimeric antibody” is an antibody which is obtained by recombination of a variable region of a mouse antibody and a constant region of a human antibody, and has a greatly improved immune response as compared with the mouse antibody.

In addition, as used herein, the term “humanized antibody” refers to an antibody obtained by modifying a protein sequence of an antibody derived from a non-human species so that the protein sequence is similar to an antibody variant naturally produced in humans. For example, the humanized antibody may be prepared as follows. Mouse-derived CDRs may be recombined with a human antibody-derived FR to prepare a humanized variable region, and the humanized variable region may be recombined with a constant region of a preferred human antibody to prepare a humanized antibody.

In the present invention, the binding molecule may be provided as a bispecific antibody or a bispecific antigen-binding fragment which is capable of binding to Lrig-1 protein and also binding to another protein.

In the present invention, the bispecific antibody and the bispecific antigen-binding fragment may comprise the binding molecule according to the present invention. As an example of the present invention, the bispecific antibody and the bispecific antigen-binding fragment comprise an antigen-binding domain capable of binding to Lrig-1 protein, wherein the antigen-binding domain capable of binding to Lrig-1 protein may comprise or consist of the binding molecule according to the present invention.

The bispecific antibody and the bispecific antigen-binding fragment provided in the present invention comprise an antigen-binding domain, which is a binding molecule capable of binding to Lrig-1 protein according to the present invention, and an antigen-binding domain capable of binding to another target protein. Here, the antigen-binding domain capable of binding another target protein may be an antigen-binding domain capable of binding to a protein other than Lrig-1 protein, for example, but not limited to, PD-1 or a cell surface receptor. However, the antigen-binding domain is not limited thereto.

The bispecific antibody and the bispecific antigen-binding fragment according to the present invention may be provided in any suitable format, for example, that described in Kontermann MAbs 2012, 4(2): 182-197, which is incorporated herein by reference in its entirety. For example, the bispecific antibody or the bispecific antigen-binding fragment may be a bispecific antibody conjugate (for example, IgG2, F(ab′)2, or CovX-body), a bispecific IgG or IgG-like molecule (for example, IgG, scFv4-Ig, IgG-scFv, scFv-IgG, DVD-Ig, IgG-sVD, sVD-IgG, or 2 in 1-IgG, mAb2, or Tandemab common LC), an asymmetric bispecific IgG or IgG-like molecule (for example, kih IgG, kih IgG common LC, CrossMab, kih IgG-scFab, mAb-Fv, charge pair, or SEED-body), a small bispecific antibody molecule (for example, diabody (Db), dsDb, DART, scDb, tandAb, tandem scFv (taFv), tandem dAbNHH, triple body, triple head, Fab-scFv, or F(ab′)2-scFv2), a bispecific Fc and CH3 fusion protein (for example, taFv-Fc, di-diabody, scDb-CH3, scFv-Fc-scFv, HCAb-VHH, scFv-kih-Fc, or scFv-kih-CH3), or a bispecific fusion protein (for example, scFv2-albumin, scDb-albumin, taFv-toxin, DNL-Fab3, DNL-Fab4-IgG, DNL-Fab4-IgG-cytokine 2). See, in particular, FIG. 2 in Kontermann MAbs 2012, 4 (2): 182-19. The bispecific antibody and the bispecific antigen-binding fragment according to the invention may be designed and prepared by those skilled in the art.

A method for producing the bispecific antibody in the present invention comprises forming a reducing disulfide or non-reducing thioether bond, and chemical crosslinking of an antibody or antibody fragment as described, for example, in Segal and Bast, 2001. Production of Bispecific Antibodies. Current Protocols in Immunology. 14:IV:2.13:2.13.1-2.13.16, which is incorporated herein by reference in its entirety. For example, N-succinimidyl-3-(-2-pyridyldithio)-propionate (SPDP) may be used, for example, for chemically crosslinking an Fab fragment through an SH-group at the hinge region, to generate a disulfide-linked bispecific F(ab)2 heterodimer.

In addition, an alternative method for producing the bispecific antibody in the present invention comprises fusing an antibody-producing hybridoma with, for example, polyethylene glycol, to produce quadroma cells capable of secreting bispecific antibodies, as described, for example, in D. M. and Bast, B. J. 2001. Production of Bispecific Antibodies. Current Protocols in Immunology. 14:IV:2.13:2.13.1-2.13.16.

The bispecific antibody and the bispecific antigen-binding fragment according to the invention may also be, for example, recombinantly produced by expression from a nucleic acid construct that encodes a polypeptide for an antigen-binding molecule, as described, for example, in Antibody Engineering: Methods and Protocols, Second Edition (Humana Press, 2012), at Chapter 40: Production of Bispecific Antibodies: Diabodies and Tandem scFv (Hornig and Farber-Schwarz), or French, How to make bispecific antibodies, Methods Mol. Med. 2000; 40:333-339, both of which are incorporated herein by reference in their entireties.

For example, a DNA construct that contains a sequence encoding light and heavy chain variable domains for two antigen-binding domains (that is, light and heavy chain variable domains for an antigen-binding domain capable of binding to PD-1, and light and heavy chain variable domains for an antigen-binding domain capable of binding to another target protein), and a sequence encoding a suitable linker or dimerization domain between the antigen-binding domains may be prepared by molecular cloning techniques. Subsequently, a recombinant bispecific antibody may be produced by expression of the construct (for example, in vitro) in a suitable host cell (for example, a mammalian host cell), and then the expressed recombinant bispecific antibody may be optionally purified.

Antibodies may be produced by an affinity maturation process in which a modified antibody with improved affinity for an antigen as compared with an unmodified parent antibody is produced. An affinity matured antibody may be produced by a procedure known in the art, for example, in Marks et al., Rio/Technology 10:779-783 (1992); Barbas et al. Proc Nat. Acad. Sci. USA 91:3809-3813 (1994); Schier et al. Gene 169:147-155 (1995); Yelton et al. J. Immunol. 155:1994-2004 (1995); Jackson et al., J. Immunol. 154 (7):3310-159 (1995); and Hawkins et al, J. Mol. Biol. 226:889-896 (1992).

In addition, the binding molecule provided in the present invention may include a variant of the amino acid sequence as long as the variant can specifically bind to Lrig-1 protein. For example, in order to improve binding affinity and/or other biological properties of an antibody, modifications may be made to an amino acid sequence of the antibody. Such modifications include, for example, deletions, insertions, and/or substitutions of amino acid sequence residues of the antibody.

Such amino acid variations are made based on relative similarity of amino acid side chain substituents such as hydrophobicity, hydrophilicity, charge, and size. According to analysis on sizes, shapes, and types of amino acid side chain substituents, it can be seen that arginine, lysine, and histidine are all positively charged residues; alanine, glycine, and serine have similar sizes; and phenylalanine, tryptophan, and tyrosine have similar shapes. Thus, based on these considerations, it can be said that arginine, lysine, and histidine; alanine, glycine, and serine; and phenylalanine, tryptophan, and tyrosine are biologically functional equivalents.

In introducing variations, the hydropathic index of amino acids may be considered. Each amino acid has been assigned hydropathic index depending on its hydrophobicity and charge: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5). The hydropathic amino acid index is very important in conferring the interactive biological function on a protein. It is known that substitution with an amino acid having similar hydropathic index allows a protein to retain similar biological activity. In a case where variations are introduced with reference to the hydropathic index, substitutions are made between amino acids that exhibit a hydropathic index difference of preferably within ±2, more preferably within ±1, and even more preferably within ±0.5.

Meanwhile, it is also well known that substitutions between amino acids having similar hydrophilicity values result in proteins with equivalent biological activity. As disclosed in U.S. Pat. No. 4,554,101, respective amino acid residues have been assigned the following hydrophilicity values: arginine (+3.0); lysine (+3.0); aspartate (+3.0±1); glutamate (+3.0±1); serine (+0.3); asparagine (+0.2); glutamine (+0.2); glycine (0); threonine (−0.4); proline (−0.5±1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). In a case where variations are introduced with reference to the hydrophilicity values, substitutions may be made between amino acids that exhibit a hydrophilicity value difference of preferably within ±2, more preferably within ±1, and even more preferably within ±0.5.

Amino acid exchanges in proteins which do not entirely alter activity of a molecule are known in the art (H. Neurath, R. L. Hill, The Proteins, Academic Press, New York (1979)). The most commonly occurring exchanges are exchanges between amino acid residues Ala/Ser, Val/Ile, Asp/Glu, Thr/Ser, Ala/Gly, Ala/Thr, Ser/Asn, Ala/Val, Ser/Gly, Tyr/Phe, Ala/Pro, Lys/Arg, Asp/Asn, Leu/Ile, Leu/Val, Gln/Glu.

Given the above-described variations with biologically equivalent activity, it is interpreted that the binding molecule of the present invention also includes sequences that exhibit substantial identity with the sequences listed in the Sequence Listing.

As used herein, the term “substantial identity” refers to a sequence showing at least 61% homology, more preferably 70% homology, even more preferably 80% homology, and most preferably 90% homology when the sequence of the present invention is aligned with any other sequence so that they maximally correspond to each other, and the aligned sequence is analyzed by using an algorithm typically used in the art. Alignment methods for comparison of sequences are known in the art. Various methods and algorithms for alignment are disclosed in Smith and Waterman, Adv. Appl. Math. 2:482 (1981); Needleman and Wunsch, J. Mol. Bio. 48:443 (1970); Pearson and Lipman, Methods in Mol. Biol. 24: 307-31 (1988); Higgins and Sharp, Gene 73:237-44 (1988); Higgins and Sharp, CABIOS 5:151-3 (1989); Corpet et al., Nuc. Acids Res. 16:10881-90 (1988); Huang et al., Comp. Appl. BioSci. 8:155-65 (1992); and Pearson et al., Meth. Mol. Biol. 24:307-31 (1994). NCBI Basic Local Alignment Search Tool (BLAST) (Altschul et al., J. Mol. Biol. 215: 403-10 (1990)) is accessible from the National Center for Biological Information (NBCI), or the like, and may be used in conjunction with sequencing programs, such as blastp, blasm, blastx, tblastn, and tblastx, on the internet. BLSAT is accessible at http://www.ncbi.nlm.nih.gov/BLAST/. Sequence homology comparison methods using this program can be identified online (http://www.ncbi.nlm.nih.gov/BLAST/blast_help.html).

In the present invention, the binding molecule, preferably the antibody, may be produced by a conventional method for producing an antibody, and may be produced by affinity maturation.

As used herein, the term “affinity maturation” refers to a process in which antibodies having increased affinity for an antigen are produced by activated B cells in the course of an immune response. For the purpose of the present invention, the affinity maturation allows antibodies or antibody fragments to be produced due to affinity maturation based on the principles of mutation and selection, in the same process that occurs in nature.

The binding molecule, preferably the antibody, provided in the present invention can effectively prevent, ameliorate, or treat brain and nervous system diseases, in particular, neurodegenerative diseases or neuroinflammatory diseases.

According to another embodiment of the present invention, there is provided a composition for preventing, ameliorating, or treating a brain and nervous system disease, comprising, as an active ingredient, a nucleic acid molecule encoding the binding molecule provided in the present invention; an expression vector into which the nucleic acid molecule is inserted; or a host cell line transfected with the expression vector.

The nucleic acid molecule of the present invention includes all nucleic acid molecules obtained by translating the amino acid sequences of the binding molecules provided in the present invention to polynucleotide sequences, as known to those skilled in the art. Therefore, various polynucleotide sequences may be prepared by an open reading frame (ORF), and all of these polynucleotide sequences are also included in the nucleic acid molecule of the present invention.

In the present invention, the “vector” is a nucleic acid molecule capable of transporting another nucleic acid linked thereto. One type of vector is a “plasmid,” which refers to circular double-stranded DNA into which an additional DNA segment can be ligated. Another type of vector is a phage vector. Yet another type of vector is a viral vector, where an additional DNA segment can be ligated into the viral genome. Certain vectors are capable of autonomous replication in a host cell into which they are introduced (for example, bacterial vectors having a bacterial origin of replication and episomal mammalian vectors). Other vectors (for example, non-episomal mammalian vectors) can be integrated into the genome of a host cell upon introduction into the host cell, and thus are replicated along with the host genome. In addition, certain vectors are capable of directing expression of genes to which they are operatively linked. Such vectors are referred to herein as “recombinant expression vectors” or simply “expression vectors.” In general, expression vectors useful in recombinant DNA techniques are often in the form of plasmids. In the present specification, “plasmid” and “vector” may be used interchangeably as the plasmid is the most commonly used form of vector.

Specific examples of the expression vector in the present invention may be selected from, but are not limited to, the group consisting of commercially widely used pCDNA vectors, F, R1, RP1, Col, pBR322, ToL, Ti vectors; cosmids; phages such as lambda, lambdoid, M13, Mu, p1 P22, Qμμ, T-even, T2, T3, T7; plant viruses. Any expression vector known, to those skilled in the art, as expression vectors can be used in the present invention, and the expression vector is selected depending on the nature of the target host cell. Introduction of a vector into a host cell may be performed by calcium phosphate transfection, viral infection, DEAE-dextran-mediated transfection, lipofectamine transfection, or electroporation. However, the present invention is not limited thereto, and those skilled in the art may adopt and use an introduction method appropriate for the expression vector and the host cell which are used. The vector may preferably contain at least one selection marker. However, the present invention is not limited thereto, and selection can be made using the vector that contains no selection marker, depending on whether or not a product is produced. The selection marker is selected depending on the target host cell, which is done using methods already known to those skilled in the art, and thus the present invention has no limitation thereon.

In order to facilitate purification of the nucleic acid molecule of the present invention, a tag sequence may be inserted into and fused to an expression vector. The tag includes, but is not limited to, hexa-histidine tag, hemagglutinin tag, myc tag, or flag tag, and any tag known to those skilled in the art which facilitates purification can be used in the present invention.

In the present invention, the “host cell” includes individual cells or cell cultures which may be or have been recipients of the vector(s) for incorporation of a polypeptide insert. The host cell includes progeny of a single host cell, and the progeny may not necessarily be completely identical (in morphology or in genomic DNA complement) to the original parent cell due to natural, accidental, or intentional mutation. The host cell includes cells transfected in vivo with the polynucleotide(s) herein.

In the present invention, the host cell may include cells of mammalian, plant, insect, fungal, or cellular origin, and may be, for example, bacterial cells such as E. coli, Streptomyces, Salmonella typhimurium; fungal cells such as yeast cells and Pichia pastoris; insect cells such as Drosophila and Spodoptera Sf9 cells; animal cells such as Chinese hamster ovary (CHO) cells, SP2/0 (mouse myeloma), human lymphoblastoid, COS, NSO (mouse myeloma), 293T, Bowes melanoma cells, HT-1080, baby hamster kidney (BHK) cells, human embryonic kidney (HEK) cells, or PERC.6 (human retinal cells); or plant cells. However, the host cell is not limited thereto, and any cell known to those skilled in the art which can be used as a host cell line is available.

According to still yet another embodiment of the present invention, there is provided a composition for preventing, ameliorating, or treating a brain and nervous system disease, comprising, as an active ingredient, an antibody-drug conjugate (ADC) comprising the antibody provided in the present invention and a drug.

As used herein, the term “antibody-drug conjugate (ADC)” refers to a form in which the drug and the antibody are chemically linked to each other without degrading biological activity of the antibody and the drug. In the present invention, the antibody-drug conjugate denotes a form in which the drug is bound to an amino acid residue at the N-terminus of the heavy and/or light chain of the antibody, specifically, a form in which the drug is bound to an α-amine group at the N-terminus of the heavy and/or light chain of the antibody.

As used herein, the term “drug” may mean any substance having a certain biological activity for a cell, which is a concept including DNA, RNA, or a peptide. The drug may be in a form which contains a reactive group capable of reacting and crosslinking with an α-amine group, and also includes a form which contains a reactive group capable of reacting and crosslinking with an α-amine group and to which a linker is linked.

In the present invention, examples of the reactive group capable of reacting and crosslinking with the α-amine group are not particularly limited in terms of type as long as the reactive group can react and crosslink with an a-amine group at the N-terminus of a heavy or light chain of an antibody. The reactive group includes all types of groups known in the art which react with an amine group. The reactive group may, for example, be any one of isothiocyanate, isocyanate, acyl azide, NHS ester, sulfonyl chloride, aldehyde, glyoxal, epoxide, oxirane, carbonate, aryl halide, imidoester, carbodiimide, anhydride, and fluorophenyl ester, but is not limited thereto.

In the present invention, the drug may include any drug regardless of type as long as the drug can treat brain and nervous system diseases, in particular, neurodegenerative diseases or neuroinflammatory diseases.

The brain and nervous system disease to be prevented, ameliorated, or treated by the composition provided in the present invention may be a neurodegenerative disease or neuroinflammatory disease.

In the present invention, the “neurodegenerative disease” may refer to a disease caused by decreased function or loss of neurons, and the “neuroinflammatory disease” may refer to a disease caused by excessive inflammatory responses in the nervous system. As a specific example, the neurodegenerative disease or neuroinflammatory disease in the present invention may be selected from, but is not limited to, the group consisting of stroke, dementia, Alzheimer's disease, Parkinson's disease, Huntington's disease, Niemann-Pick disease, multiple sclerosis, prion disease, Creutzfeldt-Jakob disease, frontotemporal dementia, dementia with Lewy bodies, amyotrophic lateral sclerosis, paraneoplastic syndrome, cortical degeneration syndrome, multiple system atrophy, progressive supranuclear palsy, nervous system autoimmune disease, spinocerebellar ataxia, inflammatory and neuropathic pain, cerebrovascular disease, spinal cord injury, and tauopathy.

In addition, the composition provided in the present invention may be used as a pharmaceutical composition or a food composition. However, the present invention is not limited thereto.

In the present invention, the “prevention” may include, without limitation, any act of blocking symptoms caused by a neurodegenerative disease or neuroinflammatory disease, or suppressing or delaying the symptoms, using the composition of the present invention.

In the present invention, the “treatment” or “amelioration” may include, without limitation, any act capable of ameliorating or beneficially altering symptoms caused by a neurodegenerative disease or neuroinflammatory disease, using the composition of the present invention.

In the present invention, the pharmaceutical composition may be characterized by being in the form of capsules, tablets, granules, injections, ointments, powders, or beverages, and the pharmaceutical composition may be characterized by being targeted to humans.

The pharmaceutical composition of the present invention may be formulated in the form of oral preparations such as powders, granules, capsules, tablets, and aqueous suspensions, preparations for external use, suppositories, and sterile injectable solutions, respectively, according to conventional methods, and used. However, the pharmaceutical composition is not limited thereto. The pharmaceutical composition of the present invention may further comprise a pharmaceutically acceptable carrier. As the pharmaceutically acceptable carrier, a binder, a glidant, a disintegrant, an excipient, a solubilizer, a dispersant, a stabilizer, a suspending agent, a pigment, a flavor, and the like may be used for oral administration; a buffer, a preserving agent, a pain-relieving agent, a solubilizer, an isotonic agent, a stabilizer, and the like may be used in admixture for injections; and a base, an excipient, a lubricant, a preserving agent, and the like may be used for topical administration. The preparations of the pharmaceutical composition of the present invention may be prepared in various ways by being mixed with the pharmaceutically acceptable carrier as described above. For example, for oral administration, the pharmaceutical composition may be formulated in the form of tablets, troches, capsules, elixirs, suspensions, syrups, wafers, or the like. For injections, the pharmaceutical composition may be formulated in the form of unit dosage ampoules or multiple dosage forms. Alternatively, the pharmaceutical composition may be formulated into solutions, suspensions, tablets, capsules, sustained-release preparations, or the like.

Meanwhile, as examples of carriers, diluents, or excipients suitable for making preparations, lactose, dextrose, sucrose, sorbitol, mannitol, xylitol, erythritol, maltitol, starch, gum acacia, alginate, gelatin, calcium phosphate, calcium silicate, cellulose, methyl cellulose, microcrystalline cellulose, polyvinylpyrrolidone, water, methyl hydroxybenzoate, propyl hydroxybenzoate, talc, magnesium stearate, mineral oil, or the like may be used. In addition, a filler, an anti-coagulant, a lubricant, a wetting agent, a fragrance, an emulsifier, a preservative, and the like may further be included.

The route of administration of the pharmaceutical composition according to the present invention includes, but is not limited to, oral, intravenous, intramuscular, intraarterial, intramedullary, intradural, intracardiac, transdermal, subcutaneous, intraperitoneal, intranasal, intestinal, topical, sublingual, or rectal route. Oral or parenteral administration is preferred.

In the present invention, the “parenteral” includes subcutaneous, intradermal, intravenous, intramuscular, intraarticular, intrabursal, intrasternal, intradural, intralesional, and intracranial injection or infusion techniques. The pharmaceutical composition of the present invention may also be administered in the form of suppositories for rectal administration.

The pharmaceutical composition of the present invention may vary depending on a variety of factors, including activity of a certain compound used, the patient's age, body weight, general health status, sex, diet, frequency of administration, route of administration, rate of excretion, drug combination, and severity of a certain disease to be prevented or treated. A dose of the pharmaceutical composition may vary depending on the patient's condition, body weight, severity of disease, drug form, route of administration, and duration, and may be appropriately selected by those skilled in the art. The pharmaceutical composition may be administered in an amount of 0.0001 to 50 mg/kg or 0.001 to 50 mg/kg, per day. Administration may be made once a day or several times a day. The dose is not intended to limit the scope of the invention in any way. The pharmaceutical composition according to the present invention may be formulated in the form of pills, sugar-coated tablets, capsules, liquids, gels, syrups, slurries, or suspensions.

The food composition that includes the composition of the present invention may be prepared in the form of various foods, for example, beverages, gums, tea, vitamin complexes, powders, granules, tablets, capsules, confections, rice cakes, bread, and the like. The food composition of the present invention is composed of a plant extract having little toxicity and side effects, and thus can be used without worries in a case of being ingested for a long time for preventive purposes.

When the composition of the present invention is included in the food composition, it may be added in an amount corresponding to a rate of 0.1% to 50% of the total weight.

Here, in a case where the food composition is prepared in the form of beverages, there is no particular limitation except that the beverage contains the food composition at an indicated proportion, and the beverage may contain various flavoring agents, natural carbohydrates, or the like as additional ingredients, similarly to conventional beverages. That is, examples of the natural carbohydrates may include monosaccharides such as glucose, disaccharides such as fructose, polysaccharides such as sucrose, conventional sugars such as dextrin and cyclodextrin, and sugar alcohol such as xylitol, sorbitol, and erythritol. Examples of the flavoring agents may include natural flavoring agents (thaumatin, stevia extracts (such as rebaudioside A), glycyrrhizin, and the like) and synthetic flavoring agents (saccharin, aspartame, and the like).

In addition, the food composition of the present invention may contain various nutrients, vitamins, minerals (electrolytes), flavorings such as synthetic flavorings and natural flavorings, colorants, pectic acid and salts thereof, alginic acid and salts thereof, organic acids, protective colloidal thickeners, pH adjusting agents, stabilizers, preservatives, glycerin, alcohol, carbonizing agents used in carbonated beverages, and the like.

These ingredients may be used individually or in combination. A proportion of such additives is not so important, and is generally selected from the range of 0.1 to about 50 parts by weight per 100 parts by weight of the composition of the present invention.

According to still yet another embodiment of the present invention, there is provided a method for preventing, ameliorating, or treating a brain and nervous system disease, comprising a step of administering, to an individual in need thereof, the binding molecule provided in the present invention; a nucleic acid molecule encoding the binding molecule; an expression vector into which the nucleic acid molecule is inserted; a host cell line transfected with the expression vector; or the antibody-drug conjugate (ADC) provided in the present invention.

In the present invention, the “individual” is an individual suspected of developing a brain and nervous system disease, and the individual suspected of developing a brain and nervous system disease means a mammal, such as mice and domestic animals, including humans, who has developed or is likely to develop the disease in question. However, any individual, who is treatable with the active substance provided in the present invention, is included therein without limitation.

The brain and nervous system disease treated by the method of the present invention may be a neurodegenerative disease or neuroinflammatory disease. As a specific example, the neurodegenerative disease or neuroinflammatory disease in the present invention may be selected from, but is not limited to, the group consisting of stroke, dementia, Alzheimer's disease, Parkinson's disease, Huntington's disease, Niemann-Pick disease, multiple sclerosis, prion disease, Creutzfeldt-Jakob disease, frontotemporal dementia, dementia with Lewy bodies, amyotrophic lateral sclerosis, paraneoplastic syndrome, cortical degeneration syndrome, multiple system atrophy, progressive supranuclear palsy, nervous system autoimmune disease, spinocerebellar ataxia, inflammatory and neuropathic pain, cerebrovascular disease, spinal cord injury, and tauopathy.

The method of the present invention may comprise a step of administering, in a pharmaceutically effective amount, the binding molecule provided in the present invention; a nucleic acid molecule encoding the binding molecule; an expression vector into which the nucleic acid molecule is inserted; a host cell line transfected with the expression vector; or the antibody-drug conjugate (ADC) provided in the present invention.

An appropriate total daily amount used may be determined by an attending physician or veterinarian within the scope of sound medical judgment, and administration may be made once or several times. However, for the purposes of the present invention, a specific therapeutically effective amount for a particular patient is preferably applied differently depending on various factors, including type and degree of reaction to be achieved, the specific composition comprising the active ingredient, including whether other agents are used therewith as the case may be, the patient's age, body weight, general health status, sex, and diet, frequency of administration, route of administration, secretion rate of the composition comprising the active ingredient, duration of treatment, and drugs used simultaneously or in combination with the specific composition, and similar factors well known in the medical field.

Meanwhile, the method for preventing or treating a brain and nervous system disease may be, but is not limited to, a combination therapy that further comprises a step of administering a compound or substance having therapeutic activity against one or more diseases.

In the present invention, the “combination” should be understood to represent simultaneous, individual, or sequential administration. In a case where the administration is made in a sequential or individual manner, the second component should be administered at intervals such that beneficial effects of the combination are not lost.

In the present invention, the dosage of the binding molecule or the antibody-drug conjugate may be, but is not limited to, about 0.0001 μg to 500 mg per kg of patient's body weight.

According to still yet another embodiment of the present invention, there is provided a diagnostic composition for a brain and nervous system disease, comprising the binding molecule provided in the present invention.

In the present invention, the “diagnostic” means identifying the presence or characteristics of a pathological condition. For the purpose of the present invention, diagnostic is intended to determine whether or not a brain and nervous system disease, in particular, a neurodegenerative disease or neuroinflammatory disease has developed.

In the present invention, it is possible to diagnose a brain and nervous system disease by measuring an expression level of Lrig-1 protein using the binding molecule.

According to still yet another embodiment of the present invention, there is provided a diagnostic kit for a brain and nervous system disease, comprising the diagnostic composition of the present invention.

In the present invention, the term “kit” refers to a set of compositions and accessories required for a specific purpose. For the purposes of the present invention, the kit of the present invention can diagnose the disease by checking an expression level of Lrig-1 protein. The kit of the present invention may include not only a primer, a probe, or an antibody, which selectively recognizes a diagnostic marker for immune related disease, for measuring an expression level of the marker, but also one or more other component compositions, solutions, or devices suitable for assay methods.

In the present invention, the kit may be, but is not limited to, an RT-PCR kit, a DNA chip kit, an ELISA kit, a protein chip kit, a rapid kit, or a multiple reaction monitoring (MRM) kit.

The diagnostic kit of the present invention may further include one or more other component compositions, solutions, or devices suitable for assay methods.

For example, the diagnostic kit of the present invention may further include essential elements required for performing reverse transcription polymerase reaction. The kit for reverse transcription polymerase reaction includes a pair of primers specific for a gene encoding a marker protein. The primer is a nucleotide having a sequence specific to a nucleic acid sequence of the gene, and may have a length of about 7 bp to 50 bp, more preferably about 10 bp to 30 bp. The kit may also include a primer specific for a nucleic acid sequence of a control gene. In addition to those mentioned above, the kit for reverse transcription polymerase reaction may include test tubes or other suitable containers, reaction buffers (with varying pH and magnesium concentrations), deoxynucleotides (dNTPs), enzymes such as Taq-polymerase and reverse transcriptase, DNase and RNase inhibitors, DEPC-treated water, sterilized water, and the like.

In addition, the diagnostic kit of the present invention may include essential elements required for performing a DNA chip assay. The DNA chip kit may include a substrate to which a cDNA or oligonucleotide corresponding to a gene or a fragment thereof is attached; reagents, agents, and enzymes for preparing a fluorescence-labeled probe; and the like. The substrate may also contain a cDNA or oligonucleotide corresponding to a control gene or a fragment thereof.

In addition, the diagnostic kit of the present invention may include essential elements required for performing ELISA. The ELISA kit includes an antibody specific for the marker protein. The antibodies are antibodies with high specificity and affinity for the marker protein and little cross-reactivity with other proteins, and include monoclonal antibodies, polyclonal antibodies, or recombinant antibodies. The ELISA kit may also include an antibody specific for a control protein. In addition to those mentioned above, the ELISA kit may include reagents capable of detecting bound antibodies, for example, labeled secondary antibodies, chromophores, enzymes (which are, for example, conjugated with an antibody) and substrates thereof, and other substances capable of binding to antibodies.

According to still yet another embodiment of the present invention, there is provided a method for providing information on diagnosis of a brain and nervous system disease, comprising a step of measuring an expression level of Lrig-1 protein present in a biological sample of a target individual by using the binding molecule provided in the present invention.

In the present invention, the “target individual” refers to an individual for whom it is uncertain whether a brain and nervous system disease, in particular, a neurodegenerative disease or neuroinflammatory disease has developed and who has a high probability of developing the disease.

In the present invention, the “biological sample” may refer to any material, tissue, cell, whole blood, serum, plasma, tissue biopsy sample (brain, skin, lymph node, spinal cord, or the like), cell culture supernatant, ruptured eukaryotic cell, bacterial expression system, or the like, which is obtained or derived from an individual, with regulatory T cell being preferred.

In the present invention, examples of the method for measuring or comparatively analyzing an expression level of Lrig-1 protein include, but are not limited to, protein chip assay, immunoassay, ligand binding assay, matrix assisted laser desorption/ionization time of flight mass spectrometry (MALDI-TOF), sulface enhanced laser desorption/ionization time of flight mass spectrometry (SELDI-TOF), radioimmunoassay, radial immunodiffusion, ouchterlony immunodiffusion, rocket immunoelectrophoresis, immunohistochemical staining, complement fixation assay, two-dimensional electrophoresis, liquid chromatography-mass spectrometry (LC-MS), liquid chromatography-mass spectrometry/mass spectrometry (LC-MS/MS), Western blotting, and enzyme linked immunosorbent assay (ELISA).

In the present invention, the method may comprise a step of predicting that the target individual has a high probability of developing a neurodegenerative disease or neuroinflammatory disease in a case where the expression level of Lrig-1 protein measured for the individual's biological sample is increased or decreased as compared with a normal control.

In the present invention, the brain and nervous system disease, which is a disease to be diagnosed through the diagnostic composition, the diagnostic kit, or the information providing method, may be a neurodegenerative disease or neuroinflammatory disease. As a specific example, the neurodegenerative disease or neuroinflammatory may be selected from, but is not limited to, the group consisting of stroke, dementia, Alzheimer's disease, Parkinson's disease, Huntington's disease, Niemann-Pick disease, multiple sclerosis, prion disease, Creutzfeldt-Jakob disease, frontotemporal dementia, dementia with Lewy bodies, amyotrophic lateral sclerosis, paraneoplastic syndrome, cortical degeneration syndrome, multiple system atrophy, progressive supranuclear palsy, nervous system autoimmune disease, spinocerebellar ataxia, inflammatory and neuropathic pain, cerebrovascular disease, spinal cord injury, and tauopathy.

### Advantageous Effects of Invention

The binding molecule provided in the present invention can not only specifically prevent, ameliorate, or treat brain and nervous system diseases such as various neurodegenerative or neuroinflammatory diseases, but also provide a method for diagnosing such diseases.

### BRIEF DESCRIPTION OF DRAWINGS

FIG. 1 illustrates a structure of the Lrig-1 protein according to an embodiment of the present invention.

FIG. 2 illustrates a structure of the Lrig-1 protein according to an embodiment of the present invention.

FIG. 3 illustrates prediction results for epitopes of the Lrig-1 protein (SEQ ID NOs: 36-57) according to an embodiment of the present invention.

FIG. 4 illustrates prediction results for epitopes of the Lrig-1 protein according to an embodiment of the present invention.

FIG. 5 illustrates an expression level of Lrig-1 mRNA according to an embodiment of the present invention.

FIG. 6 illustrates an expression level of Lrig-1 mRNA according to an embodiment of the present invention.

FIG. 7 illustrates an expression level of Lrig-1 mRNA according to an embodiment of the present invention.

FIG. 8 illustrates expression levels of Lrig-1, Lrig-2, and Lrig-3 mRNAs according to an embodiment of the present invention.

FIG. 9 illustrates results obtained by comparing expression levels of Lrig-1 protein in regulatory T cells and non-regulated T cells according to an embodiment of the present invention.

FIG. 10 illustrates expression of the Lrig-1 protein on the surface of regulatory T cells according to an embodiment of the present invention.

FIG. 11 illustrates results obtained by analyzing binding capacity of Lrig-1 protein-specific monoclonal antibodies to the Lrig-1 protein according to an embodiment of the present invention.

FIG. 12 illustrates an experimental design for identifying therapeutic effects of Lrig-1 protein-specific monoclonal antibodies on multiple sclerosis, according to an embodiment of the present invention.

FIG. 13 illustrates results obtained by administering Lrig-1 protein-specific monoclonal antibodies, and then analyzing their therapeutic effects on multiple sclerosis, according to an embodiment of the present invention.

FIG. 14 illustrates results obtained by administering Lrig-1 protein-specific monoclonal antibodies, and then analyzing their therapeutic effects on Alzheimer's disease, according to an embodiment of the present invention.

FIG. 15 illustrates results obtained by administering Lrig-1 protein-specific monoclonal antibodies, and then analyzing their therapeutic effects on Alzheimer's disease, according to an embodiment of the present invention.

FIG. 16 illustrates results obtained by administering Lrig-1 protein-specific monoclonal antibodies, and then analyzing their therapeutic effects on Alzheimer's disease, according to an embodiment of the present invention.

FIG. 17 illustrates results obtained by administering Lrig-1 protein-specific monoclonal antibodies, and then analyzing their therapeutic effects on Alzheimer's disease, according to an embodiment of the present invention.

### DETAILED DESCRIPTION OF INVENTION

In the present invention, there is provided a composition for preventing, ameliorating, or treating a brain and nervous system disease, comprising, as an active ingredient, a binding molecule that specifically binds to leucine-rich and immunoglobulin-like domains 1 (Lrig-1) protein present on the surface of regulatory T cells (Treg cells)

Here, the binding molecule may be a binding molecule, comprising:

a heavy chain variable region that contains a heavy chain CDR1 consisting of the amino acid sequence represented by SEQ ID NO: 5 or 13, a heavy chain CDR2 consisting of the amino acid sequence represented by SEQ ID NO: 6 or 14, and a heavy chain CDR3 consisting of the amino acid sequence represented by SEQ ID NO: 7 or 15; and

a light chain variable region that contains a light chain CDR1 consisting of the amino acid sequence represented by SEQ ID NO: 8 or 16, a light chain CDR2 consisting of the amino acid sequence represented by SEQ ID NO: 9 or 17, and a light chain CDR3 consisting of the amino acid sequence represented by SEQ ID NO: 10 or 18.

Hereinafter, the present invention will be described in more detail by way of examples. These examples are only for describing the present invention in more detail, and it will be apparent to those skilled in the art that according to the gist of the present invention, the scope of the present invention is not limited by these examples.

### EXAMPLES

### Preparation Example 1 T Cell Subset Cell Culture

In order to identify whether the Lrig-1 protein is expressed only in regulatory T cells (Treg), the subsets of T cells, Th0, Th1, Th2, Th17, and iTreg, were prepared. The iTreg refers to cells whose differentiation has been artificially induced in a medium containing the following composition, unlike nTreg which has been naturally isolated.

The subsets of the T cells were induced to differentiate into respective cells by first isolating naive T cells obtained from the spleen of mice, causing RPMI1640 (Invitrogen Gibco, Grand Island, N.Y.) nutrient medium that contains 10% fetal bovine serum (FBS; HyClone, Logan, Utah) to further contain the respective ingredients of Table 1 below, and performing 72-hour incubation in an incubator at 37° C., 5% CO₂.

TABLE 1

| Differentiated cell | Composition |
|---|---|
| Th0 | anti-CD3, anti-CD28 |
| Th1 | IL-12, anti-IL-4 antibody |
| Th2 | IL-4, anti-IFNβ |
| Th17 | IL-6, TGFβ, anti-IFNβ, anti-IL-4 |
| iTreg | IL-2, TGFβ |

### Example 1 Structural Analysis of Lrig-1

A three-dimensional steric structure of the extracellular domain of the Lrig-1 protein was predicted to produce antibodies specific for the Lrig-1 protein, a surface protein of regulatory T cells.

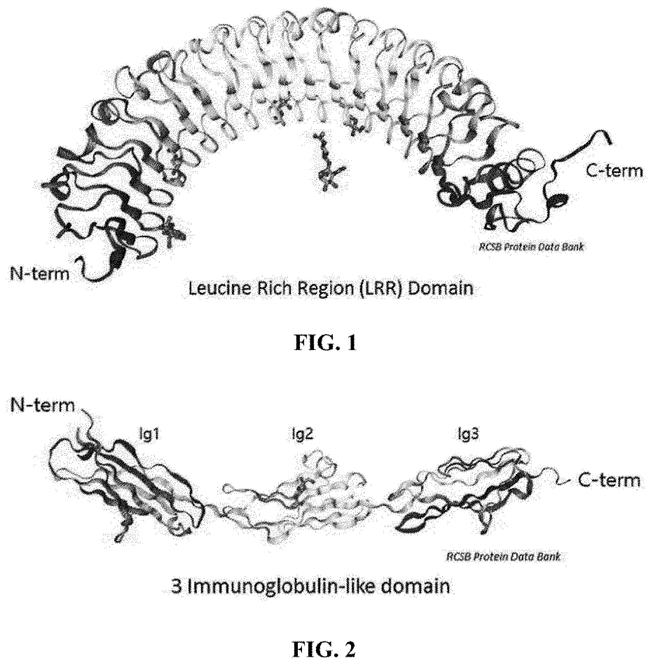

First, in order to predict base sequences of epitopes (epitopes), tools of Uniprot (http://www.uniprot.org) and RCSB Protein Data Bank (http://www.rcsb.org/pdb) were used to predict a three-dimensional steric structure of the extracellular domain (ECD) of the Lrig-1 protein so that the structure of ECD is identified. Then, the results are illustrated in FIGS. 1 and 2.

As illustrated in FIG. 1, a total of 15 leucine-rich regions of LRR1 to LRR15 existed in the Lrig-LRR domain (amino acid sequence at positions 41 to 494) in the extracellular domain of the Lrig-1 protein. Each of the LRR domains is composed of 23 to 27 amino acids, with 3 to 5 leucine being present.

In addition, as illustrated in FIG. 2, three immunoglobulin-like domains exist in amino acid sequences at positions 494 to 781 of the Lrig-1 protein in the extracellular domain of the Lrig-1 protein.

### Example 2 Prediction of Lrig-1 Epitope Amino Acid Sequence

Prediction of the above base sequence was performed using Ellipro server (http://tools.iedb.org/ellipro/) which is an epitope prediction software based on a structure of the Lrig-1 protein. The Ellipro search engine was used because it corresponds to a search engine known to be the most reliable among the existing algorithms for predicting an epitope.

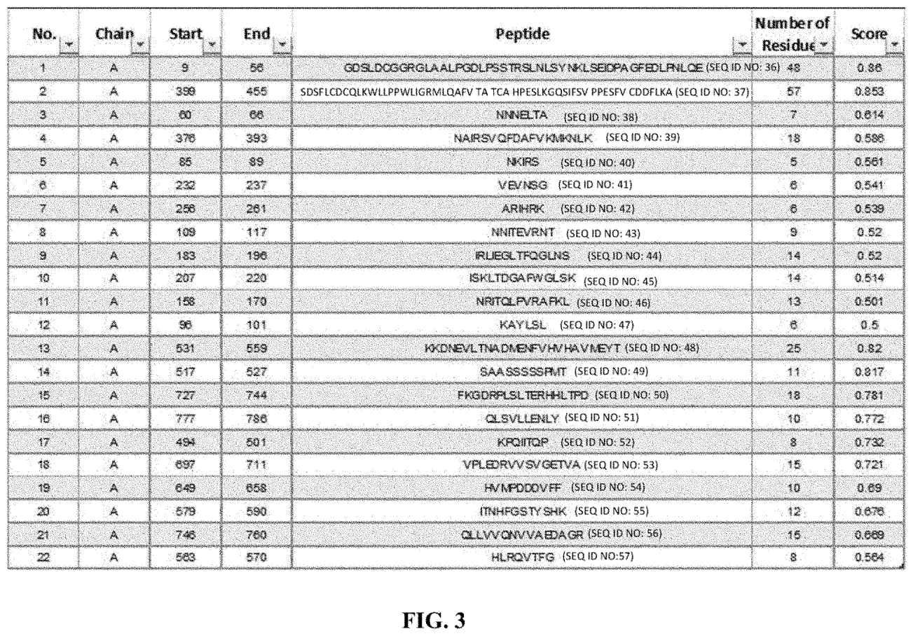

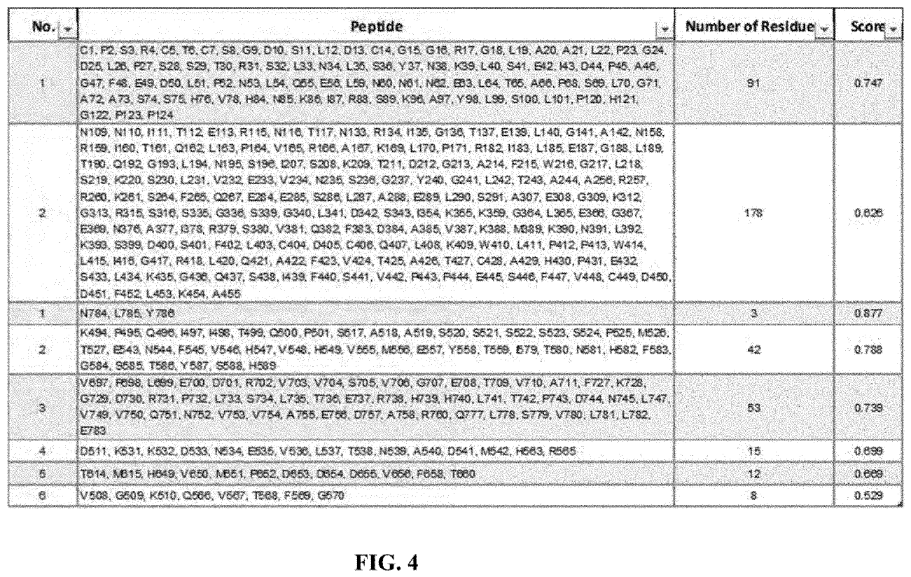

The extracellular domain analyzed in Example 1 was entered into the epitope prediction software, and then predicted contiguous or discontiguous amino acid sequences of the predicted epitopes are illustrated in FIGS. 3 and 4.

As illustrated in FIGS. 3 and 4, a total of 22 contiguous epitope amino acid sequences were predicted, and a total of 8 discontiguous epitope amino acid sequences were predicted.

### Production Examples 1 to 8 Production of Monoclonal Antibodies Specific to Lrig-1 Protein

Antibodies specific for the Lrig-1 protein according to the present invention were produced. The present antibodies were not produced by specifying a certain epitope, but were produced as antibodies capable of binding to any site on the Lrig-1 protein.

In order to produce the antibodies, cells expressing the Lrig-1 protein were produced. More specifically, a DNA fragment corresponding to SEQ ID NO: 2 and pcDNA (hygro) were cleaved with a cleavage enzyme, incubated at 37° C., and ligated to produce pcDNA into which a DNA sequence of the Lrig-1 protein is inserted. The thus produced pcDNA into which SEQ ID NO: 2 is inserted was introduced, through transfection, into L cells, so that the Lrig-1 protein is allowed to be expressed on the surface of the L cells.

Light and heavy chain amino acid sequences capable of binding to Lrig-1 expressed on the cell surface were selected from the Human scFv library so that a total of eight heavy and light chains were selected.

The selected heavy and light chain amino acid sequences were fused with the mlgG2a Fc region, to produce monoclonal antibodies. The sequences of the monoclonal antibodies are shown in Table 2 below.

TABLE 2

| | | | | Sequence |
|---|---|---|---|---|
| Classification | Clone | Location | Amino acid sequence | information |
| Production | GTC210-01 | Heavy | EVQLLESGGGLVQPGGSLRLSCAASGFTFSGY | SEQ ID NO: |
| Example 1 | clone | chain | DMSWVRQAPGKGLEWVSLIYPDSGNKYYAD | 30 |
| | | | SVKGRFTISRDNSKNTLYLQMNSLRAEDTAVY | |
| | | | YCARDAGLSWAGAFDYWGQGTLVTVSSAST | |
| | | | TAPSVYPLAPVCGDTTGSSVTLGCLVKGYFPE | |
| | | | PVTLTWNSGSLSSGVHTFPAVLQSDLYTLSSSV | |
| | | | TVTSSTWPSQSITCNVAHPASSTKVDKKIEPRG | |
| | | | PTIKPCPPCKCPAPNLLGGPSVFIFPPKIKDVLM | |
| | | | ISLSPIVTCVVVDVSEDDPDVQISWFVNNVEV | |
| | | | HTAQTQTHREDYNSTLRVVSALPIQHQDWMS | |
| | | | GKEFKCKVNNKDLPAPIERTISKPKGSVRAPQ | |
| | | | VYVLPPPEEEMTKKQVTLTCMVTDFMPEDIY | |
| | | | VEWTNNGKTELNYKNTEPVLDSDGSYFMYS | |
| | | | KLRVEKKNWVERNSYSCSVVHEGLHNHHTT | |
| | | | KSFSRTPGK- | |
| | | Light chain | QSVLTQPPSASGTPGQRVTISCSGSSSNIGSNY | SEQ ID NO: |
| | | | VTWYQQLPGTAPKLLIYSDSHRPSGVPDRFSG | 31 |
| | | | SKSGTSASLAISGLQSEDEADYYCGSWDYSLS | |
| | | | AYVFGGGTKLTVLRTVAAPTVSIFPPSSEQLTS | |
| | | | GGASVVCFLNNFYPKDINVKWKIDGSERQNG | |
| | | | VLNSWTDQDSKDSTYSMSSTLTLTKDEYERH | |
| | | | NSYTCEATHKTSTSPIVKSFNRNEC- | |
| Production | GTC210-02 | Heavy | EVQLLESGGGLVQPGGSLRLSCAASGFTFSNY | SEQ ID NO: |
| Example 2 | clone | chain | YMSWVRQAPGKGLEWVSGISPGDSSTYYADS | 58 |
| | | | VKGRFTISRDNSKNTLYLQMNSLRAEDTAVY | |
| | | | YCAKGLYSNPNEPFDYWGQGTLVTVSSASTT | |
| | | | APSVYPLAPVCGDTTGSSVTLGCLVKGYFPEP | |
| | | | VTLTWNSGSLSSGVHTFPAVLQSDLYTLSSSV | |
| | | | TVTSSTWPSQSITCNVAHPASSTKVDKKIEPRG | |
| | | | PTIKPCPPCKCPAPNLLGGPSVFIFPPKIKDVLM | |
| | | | ISLSPIVTCVVVDVSEDDPDVQISWFVNNVEV | |
| | | | HTAQTQTHREDYNSTLRVVSALPIQHQDWMS | |
| | | | GKEFKCKVNNKDLPAPIERTISKPKGSVRAPQ | |
| | | | VYVLPPPEEEMTKKQVTLTCMVTDFMPEDIY | |
| | | | VEWTNNGKTELNYKNTEPVLDSDGSYFMYS | |
| | | | KLRVEKKNWVERNSYSCSVVHEGLHNHHTT | |
| | | | KSFSRTPGK- | |
| | | Light chain | QSVLTQPPSASGTPGQRVTISCTGSSSNIGSNY | SEQ ID NO: |
| | | | VSWYQQLPGTAPKLLIYDDSQRPSGVPDRFSG | 59 |
| | | | SKSGTSASLAISGLRSEDEADYYCGTWDYSL | |
| | | | NGYVFGGGTKLTVLRTVAAPTVSIFPPSSEQLT | |
| | | | SGGASVVCFLNNFYPKDINVKWKIDGSERQN | |
| | | | GVLNSWTDQDSKDSTYSMSSTLTLTKDEYER | |
| | | | HNSYTCEATHKTSTSPIVKSFNRNEC- | |
| Production | GTC210-03 | Heavy | EVQLLESGGGLVQPGGSLRLSCAASGFTFSSY | SEQ ID NO: |
| Example 3 | clone | chain | DMSWVRQAPGKGLEWVSGISPDGSNIYYADS | 60 |
| | | | VKGRFTISRDNSKNTLYLQMNSLRAEDTAVY | |
| | | | YCAKVGLRCRYEACSYAYGMDVWGQGTLVT | |
| | | | VSSASTTAPSVYPLAPVCGDTTGSSVTLGCLV | |
| | | | KGYFPEPVTLTWNSGSLSSGVHTFPAVLQSDL | |
| | | | YTLSSSVTVTSSTWPSQSITCNVAHPASSTKVD | |
| | | | KKIEPRGPTIKPCPPCKCPAPNLLGGPSVFIFPP | |
| | | | KIKDVLMISLSPIVTCVVVDVSEDDPDVQISW | |
| | | | FVNNVEVHTAQTQTHREDYNSTLRVVSALPI | |
| | | | QHQDWMSGKEFKCKVNNKDLPAPIERTISKP | |
| | | | KGSVRAPQVYVLPPPEEEMTKKQVTLTCMVT | |
| | | | DFMPEDIYVEWTNNGKTELNYKNTEPVLDSD | |
| | | | GSYFMYSKLRVEKKNWVERNSYSCSVVHEG | |
| | | | LHNHHTTKSFSRTPGK- | |
| | | Light chain | QSVLTQPPSASGTPGQRVTISCSGSSSNIGSNY | SEQ ID NO: |
| | | | VSWYQQLPGTAPKLLIYSDSHRPSGVPDRFSG | 61 |
| | | | SKSGTSASLAISGLRSEDEADYYCATWDSSLN | |
| | | | GYVFGGGTKLTVLRTVAAPTVSIFPPSSEQLTS | |
| | | | GGASVVCFLNNFYPKDINVKWKIDGSERQNG | |
| | | | VLNSWTDQDSKDSTYSMSSTLTLTKDEYERH | |
| | | | NSYTCEATHKTSTSPIVKSFNRNEC- | |
| Production | GTC210-04 | Heavy | EVQLLESGGGLVQPGGSLRLSCAASGFTFSNY | SEQ ID NO: |
| Example 4 | clone | chain | DMSWVRQAPGKGLEWVSSISPSSGSIYYADS | 62 |
| | | | VKGRFTISRDNSKNTLYLQMNSLRAEDTAVY | |
| | | | YCAKDLDAFWRPSFDYWGQGTLVTVSSASTT | |
| | | | APSVYPLAPVCGDTTGSSVTLGCLVKGYFPEP | |
| | | | VTLTWNSGSLSSGVHTFPAVLQSDLYTLSSSV | |
| | | | TVTSSTWPSQSITCNVAHPASSTKVDKKIEPRG | |
| | | | PTIKPCPPCKCPAPNLLGGPSVFIFPPKIKDVLM | |
| | | | ISLSPIVTCVVVDVSEDDPDVQISWFVNNVEV | |
| | | | HTAQTQTHREDYNSTLRVVSALPIQHQDWMS | |
| | | | GKEFKCKVNNKDLPAPIERTISKPKGSVRAPQ | |
| | | | VYVLPPPEEEMTKKQVTLTCMVTDFMPEDIY | |
| | | | VEWTNNGKTELNYKNTEPVLDSDGSYFMYS | |
| | | | KLRVEKKNWVERNSYSCSVVHEGLHNHHTT | |
| | | | KSFSRTPGK- | |
| | | Light chain | QSVLTQPPSASGTPGQRVTISCTGSSSNIGNNN | SEQ ID NO: |
| | | | VNWYQQLPGTAPKLLIYSDSHRPSGVPDRFSG | 63 |
| | | | SKSGTSASLAISGLRSEDEADYYCGSWDDSLS | |
| | | | AYVFGGGTKLTVLRTVAAPTVSIFPPSSEQLTS | |
| | | | GGASVVCFLNNFYPKDINVKWKIDGSERQNG | |
| | | | VLNSWTDQDSKDSTYSMSSTLTLTKDEYERH | |
| | | | NSYTCEATHKTSTSPIVKSFNRNEC- | |
| Production | GTC110-01 | Heavy | EVQLLESGGGLVQPGGSLRLSCAASGFTFSDY | SEQ ID NO: |
| Example 5 | clone | chain | DMSWVRQVPGKGLEWVSWISHGGGSIYYAD | 64 |
| | | | SVKGRFTISRDNSKNTLYLQMNSLRAEDTAVY | |
| | | | YCARGLGLCKTGLCYYYDAMDVWGQGTLV | |
| | | | TVSSASTTAPSVYPLAPVCGDTTGSSVTLGCL | |
| | | | VKGYFPEPVTLTWNSGSLSSGVHTFPAVLQSD | |
| | | | LYTLSSSVTVTSSTWPSQSITCNVAHPASSTKV | |
| | | | DKKIEPRGPTIKPCPPCKCPAPNLLGGPSVFIFP | |
| | | | PKIKDVLMISLSPIVTCVVVDVSEDDPDVQIS | |
| | | | WFVNNVEVHTAQTQTHREDYNSTLRVVSALP | |
| | | | IQHQDWMSGKEFKCKVNNKDLPAPIERTISKP | |
| | | | KGSVRAPQVYVLPPPEEEMTKKQVTLTCMVT | |
| | | | DFMPEDIYVEWTNNGKTELNYKNTEPVLDSD | |
| | | | GSYFMYSKLRVEKKNWVERNSYSCSVVHEG | |
| | | | LHNHHTTKSFSRTPGK- | |
| | | Light chain | QSVLTQPPSASGTPGQRVTISCTGSSSNIGNNS | SEQ ID NO: |
| | | | VTWYQQLPGTAPKLLIYADNNRPSGVPDRFS | 65 |
| | | | GSKSGTSASLAISGLRSEDEADYYCAAWDSSL | |
| | | | SAYVFGGGTKLTVLRTVAAPTVSIFPPSSEQLT | |
| | | | SGGASVVCFLNNFYPKDINVKWKIDGSERQN | |
| | | | GVLNSWTDQDSKDSTYSMSSTLTLTKDEYER | |
| | | | HNSYTCEATHKTSTSPIVKSFNRNEC- | |
| Production | GTC110-02 | Heavy | EVQLLESGGGLVQPGGSLRLSCAASGFTFSDY | SEQ ID NO: |
| Example 6 | clone | chain | YMSWVRQAPGKGLEWVSGISHDSGSKYYAD | 66 |
| | | | SVKGRFTISRDNSKNTLYLQMNSLRAEDTAVY | |
| | | | YCARHWTTFDYWGQGTLVTVSSASTTAPSVY | |
| | | | PLAPVCGDTTGSSVTLGCLVKGYFPEPVTLTW | |
| | | | NSGSLSSGVHTFPAVLQSDLYTLSSSVTVTSST | |
| | | | WPSQSITCNVAHPASSTKVDKKIEPRGPTIKPC | |
| | | | PPCKCPAPNLLGGPSVFIFPPKIKDVLMISLSPI | |
| | | | VTCVVVDVSEDDPDVQISWFVNNVEVHTAQT | |
| | | | QTHREDYNSTLRVVSALPIQHQDWMSGKEFK | |
| | | | CKVNNKDLPAPIERTISKPKGSVRAPQVYVLP | |
| | | | PPEEEMTKKQVTLTCMVTDFMPEDIYVEWTN | |
| | | | NGKTELNYKNTEPVLDSDGSYFMYSKLRVEK | |
| | | | KNWVERNSYSCSVVHEGLHNHHTTKSFSRTP | |
| | | | GK- | |
| | | Light chain | QSVLTQPPSASGTPGQRVTISCSGSSSNIGSNN | SEQ ID NO: |
| | | | VTWYQQLPGTAPKLLIYANSNRPSGVPDRFSG | 67 |
| | | | SKSGTSASLAISGLRSEDEADYYCGAWDYSLS | |
| | | | AYVFGGGTKLTVLRTVAAPTVSIFPPSSEQLTS | |
| | | | GGASVVCFLNNFYPKDINVKWKIDGSERQNG | |
| | | | VLNSWTDQDSKDSTYSMSSTLTLTKDEYERH | |
| | | | NSYTCEATHKTSTSPIVKSFNRNEC- | |
| Production | GTC110-03 | Heavy | EVQLLESGGGLVQPGGSLRLSCAASGFTFSNY | SEQ ID NO: |
| Example 7 | clone | chain | AMSWVRQAPGKGLEWVSAIYPGGGSIYYADS | 68 |
| | | | VKGRFTISRDNSKNTLYLQMNSLRAEDTAVY | |
| | | | YCARDILPCPWGRCYYDYAMDVWGQGTLVT | |
| | | | VSSASTTAPSVYPLAPVCGDTTGSSVTLGCLV | |
| | | | KGYFPEPVTLTWNSGSLSSGVHTFPAVLQSDL | |
| | | | YTLSSSVTVTSSTWPSQSITCNVAHPASSTKVD | |
| | | | KKIEPRGPTIKPCPPCKCPAPNLLGGPSVFIFPP | |
| | | | KIKDVLMISLSPIVTCVVVDVSEDDPDVQISW | |
| | | | FVNNVEVHTAQTQTHREDYNSTLRVVSALPI | |
| | | | QHQDWMSGKEFKCKVNNKDLPAPIERTISKP | |
| | | | KGSVRAPQVYVLPPPEEEMTKKQVTLTCMVT | |
| | | | DFMPEDIYVEWTNNGKTELNYKNTEPVLDSD | |
| | | | GSYFMYSKLRVEKKNWVERNSYSCSVVHEG | |
| | | | LHNHHTTKSFSRTPGK- | |
| | | Light chain | QSVLTQPPSASGTPGQRVTISCSDSSSNIGSNT | SEQ ID NO: |
| | | | VSWYQQLPGTAPKLLIYADNNRPSGVPDRFS | 69 |
| | | | GSKSGTSASLAISGLRSEDEADYYCGTWDYS | |
| | | | LSGYVFGGGTKLTVLRTVAAPTVSIFPPSSEQL | |
| | | | TSGGASVVCFLNNFYPKDINVKWKIDGSERQ | |
| | | | NGVLNSWTDQDSKDSTYSMSSTLTLTKDEYE | |
| | | | RHNSYTCEATHKTSTSPIVKSFNRNEC- | |
| Production | GTC110-04 | Heavy | EVQLLESGGGLVQPGGSLRLSCAASGFTFSNY | SEQ ID NO: |
| Example 8 | clone | chain | AMSWVRQAPGKGLEWVSVISHGGGSTYYAD | 32 |
| | | | SVKGRFTISRDNSKNTLYLQMNSLRAEDTAVY | |
| | | | YCARVISNCHLGVCYYSNGMDVWGQGTLVT | |
| | | | VSSASTTAPSVYPLAPVCGDTTGSSVTLGCLV | |
| | | | KGYFPEPVTLTWNSGSLSSGVHTFPAVLQSDL | |
| | | | YTLSSSVTVTSSTWPSQSITCNVAHPASSTKVD | |
| | | | KKIEPRGPTIKPCPPCKCPAPNLLGGPSVFIFPP | |
| | | | KIKDVLMISLSPIVTCVVVDVSEDDPDVQISW | |
| | | | FVNNVEVHTAQTQTHREDYNSTLRVVSALPI | |
| | | | QHQDWMSGKEFKCKVNNKDLPAPIERTISKP | |
| | | | KGSVRAPQVYVLPPPEEEMTKKQVTLTCMVT | |
| | | | DFMPEDIYVEWTNNGKTELNYKNTEPVLDSD | |
| | | | GSYFMYSKLRVEKKNWVERNSYSCSVVHEG | |
| | | | LHNHHTTKSFSRTPGK- | |
| | | Light chain | QSVLTQPPSASGTPGQRVTISCSGSSSNIGNND | SEQ ID NO: |
| | | | VYWYQQLPGTAPKLLIYSDSQRPSGVPDRFSG | 33 |
| | | | SKSGTSASLAISGLRSEDEADYYCGTWDYSLS | |
| | | | GYVFGGGTKLTVLRTVAAPTVSIFPPSSEQLTS | |
| | | | GGASVVCFLNNFYPKDINVKWKIDGSERQNG | |
| | | | VLNSWTDQDSKDSTYSMSSTLTLTKDEYERH | |
| | | | NSYTCEATHKTSTSPIVKSFNRNEC- | |
| Production | GTC110-04 | Heavy | EVQLLESGGGLVQPGGSLRLSCAASGFTFSNY | SEQ ID NO: |
| Example 9 | humanized | chain | AMSWVRQAPGKGLEWVSVISHGGGSTYYAD | 34 |
| | antibody | | SVKGRFTISRDNSKNTLYLQMNSLRAEDTAVY | |
| | | | YCARVISNCHLGVCYYSNGMDVWGQGTLVT | |
| | | | VSSASTKGPSVFPLAPSSKSTSGGTAALGCLV | |
| | | | KDYFPEPVTVSWNSGALTSGVHTFPAVLQSSG | |
| | | | LYSLSSVVTVPSSSLGTQTYICNVNHKPSNTK | |
| | | | VDKKVEPKSCDKTHTCPPCPAPELLGGPSVFL | |
| | | | FPPKPKDTLMISRTPEVTCVVVDVSHEDPEVK | |
| | | | FNWYVDGVEVHNAKTKPREEQYNSTYRVVS | |
| | | | VLTVLHQDWLNGKEYKCKVSNKALPAPIEKT | |
| | | | ISKAKGQPREPQVYTLPPSREEMTKNQVSLTC | |
| | | | LVKGFYPSDIAVEWESNGQPENNYKTTPPVLD | |
| | | | SDGSFFLYSKLTVDKSRWQQGNVFSCSVMHE | |
| | | | ALHNHYTQKSLSLSPGKY | |
| | | Light chain | QSVLTQPPSASGTPGQRVTISCSGSSSNIGNND | SEQ ID NO: |
| | | | VYWYQQLPGTAPKLLIYSDSQRPSGVPDRFSG | 35 |
| | | | SKSGTSASLAISGLRSEDEADYYCGTWDYSLS | |
| | | | GYVFGGGTKLTVLRTVAAPSVFIFPPSDEQLKS | |
| | | | GTASVVCLLNNFYPREAKVQWKVDNALQSG | |
| | | | NSQESVTEQDSKDSTYSLSSTLTLSKADYEKH | |
| | | | KVYACEVTHQGLSSPVTKSFNRGEC | |

### Example 3 Identification of Specific Expression of Lrig-1 mRNA in Regulatory T Cells

Verification was made of whether the Lrig-1 protein can act as a biomarker specific for regulatory T cells.

For the verification, CD4⁺ T cells were isolated using magnet-activated cell sorting (MACS), through CD4 beads, from the spleen of mice. Subsequently, regulatory T (CD4⁺CD25⁺ T) cells and non-regulatory T (CD4⁺CD25⁻ T) cells were isolated with a fluorescence-activated cell sorter (FACS) using a CD25 antibody. For the respective cells and the cells differentiated in Preparation Example 1, mRNA was extracted using Trizol, and gDNA was removed from genomic RNA using gDNA extraction kit (Qiagen) according to the protocol provided by the manufacturer. The gDNA-removed mRNA was synthesized into cDNA through the BDsprint cDNA Synthesis Kit (Clonetech).

Real-time polymerase chain reaction (RT PCR) was performed to quantitatively identify an expression level of Lrig-1 mRNA in the cDNA.

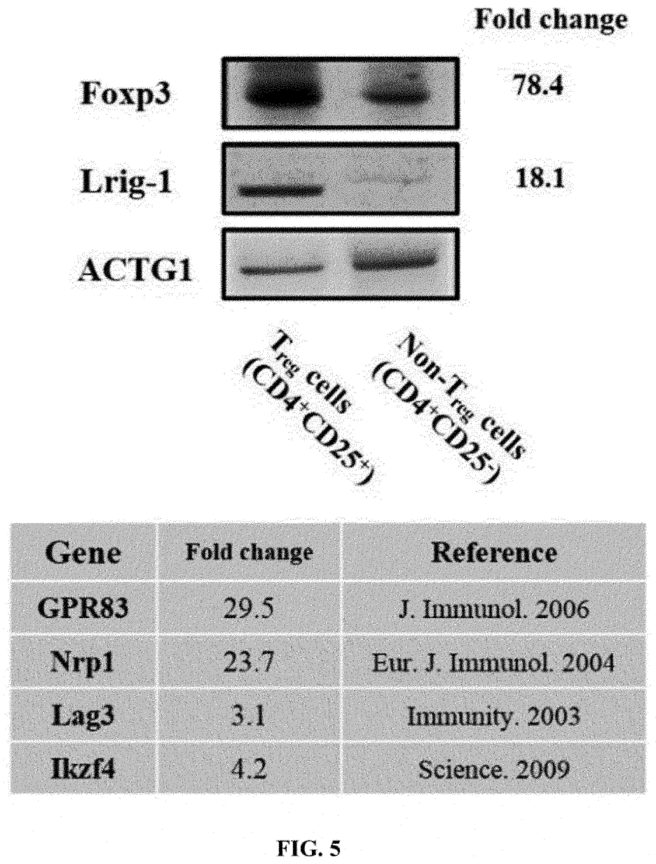

The real-time polymerase chain reaction was performed with primers shown in Table 3 below using SYBR Green (Molecular Probes) by the protocol provided by the manufacturer under conditions of 40 cycles consisting of 95° C. for 3 minutes, 61° C. for 15 seconds, 72° C. for 30 seconds, a relative gene expression level was calculated using the ΔCT method, and normalized using HPRT. The results are illustrated in FIGS. 5 to 8.

TABLE 3

| Primer | Sequence |
|---|---|
| Mouse Lrig-1 | Forward 5′- GAC GGA ATT CAG TGA GGA GAA CCT - 3′(SEQ ID NO: 70) |
| | Reverse 5′- CAA CTG GTA GTG GCA GCT TGT AGG - 3′(SEQ ID NO: 71) |
| Mouse Lrig-2 | forward 5′- TCA CAA GGA ACA TTG TCT GAA CCA - 3′(SEQ ID NO: 72) |
| | reverse 5′- GCC TGA TCT AAC ACA TCC TCC TCA - 3′(SEQ ID NO: 73) |
| Mouse Lrig-3 | forward 5′- CAG CAC CTT GAG CTG AAC AGA AAC - 3′(SEQ ID NO: 74) |
| | reverse 5′- CCA GCC TTT GGT AAT CTC GGT TAG - 3′(SEQ ID NO: 75) |
| Mouse FOXP3 | forward 5′- CTT TCA CCT ATC CCA CCC TTA TCC - 3′(SEQ ID NO: 76) |
| | reverse 5′- ATT CAT CTA CGG TCC ACA CTG CTC - 3′(SEQ ID NO: 77) |
| ACTG1 | forward 5′- GGC GTC ATG GTG GGC ATG GG - 3′(SEQ ID NO: 78) |
| | reverse 5′- ATG GCG TGG GGA AGG GCG TA - 3′(SEQ ID NO: 79) |

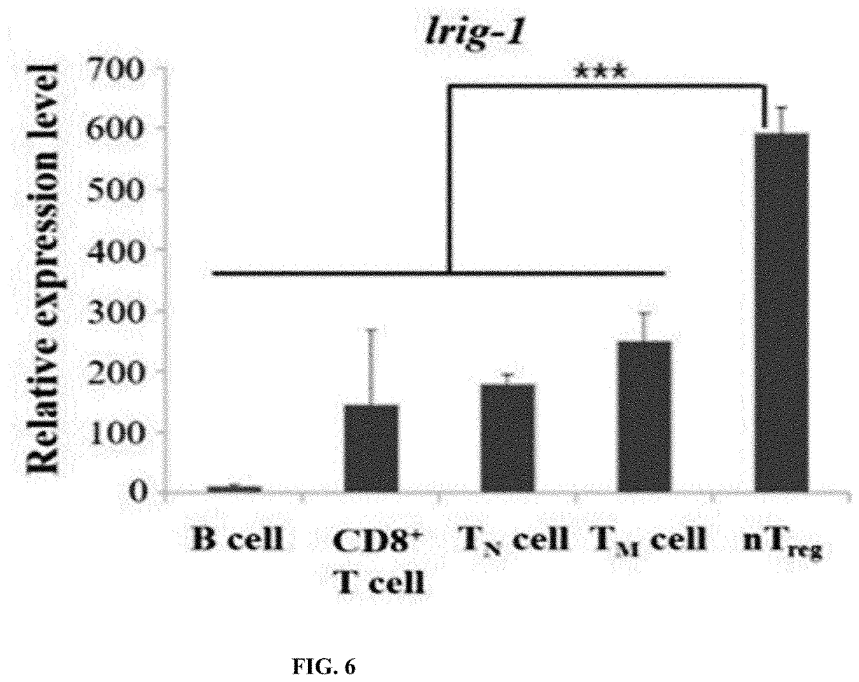

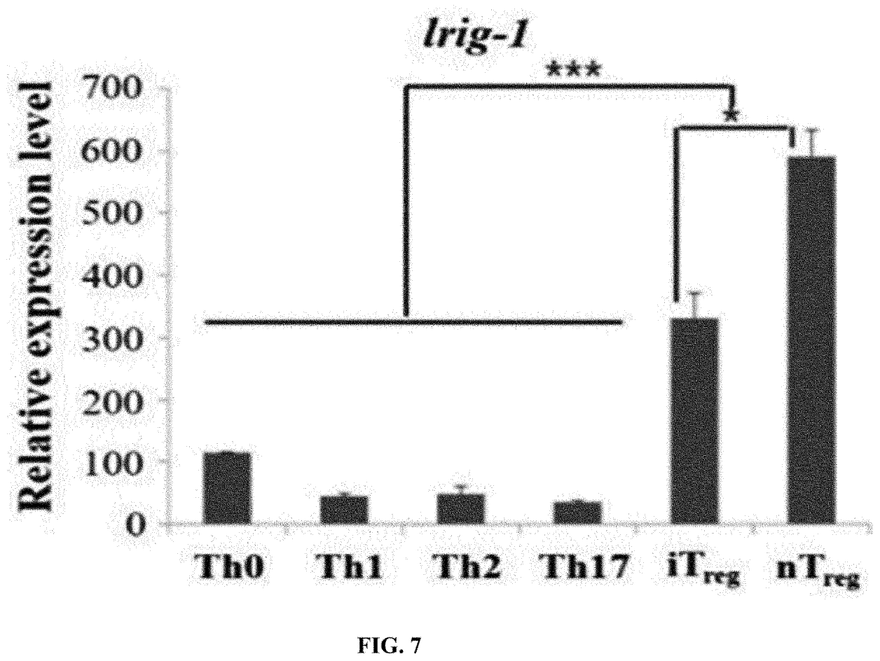

As illustrated in FIG. 5, it can be seen that the expression of Lrig-1 in regulatory T (CD4⁺CD25⁺ T) cells is 18.1 times higher than non-regulatory T (CD4⁺CD25⁻ T) cells. This was about 10 times higher expression level than Lag3 and Ikzf4, which are previously known markers for regulatory T cells. In addition, as illustrated in FIGS. 6 and 7, the expression of Lrig-1 mRNA was remarkably high in regulatory T cells as compared with other types of immune cells, and in particular, was remarkably high in naturally isolated regulatory T cells (nTreg) as compared with induced regulatory T cells (iTreg cells).

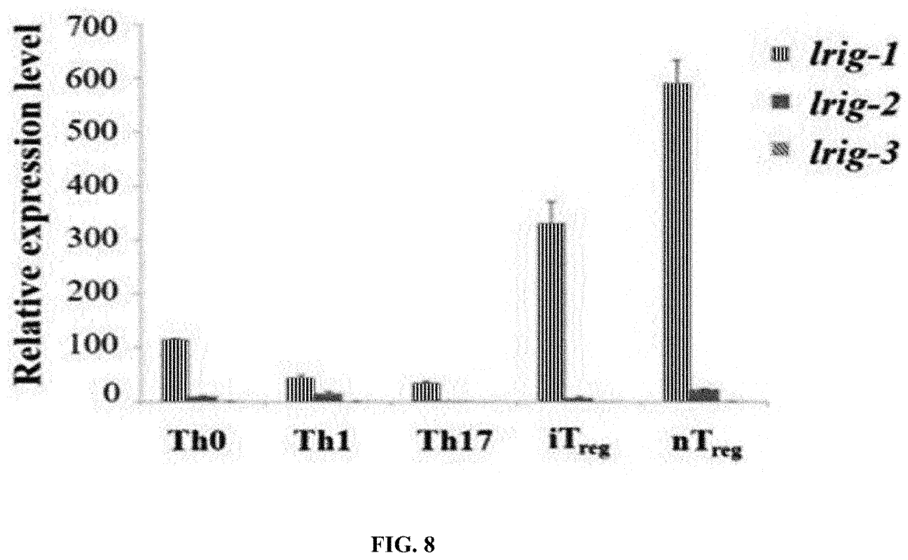

In addition, as illustrated in FIG. 8, expression of Lrig-1 was the highest among Lrig-1, Lrig-2, and Lrig-3 which correspond to the Lrig family.

From the above results, it can be seen that the Lrig-1 protein according to the present invention is specifically expressed in regulatory T cells, in particular, naturally-occurring regulatory T cells.

### Example 4 Identification of Specific Expression of Lrig-1 Protein in Regulatory T Cells

It was identified whether the Lrig-1 protein expressed from Lrig-1 mRNA is specifically expressed only in regulatory T cells.

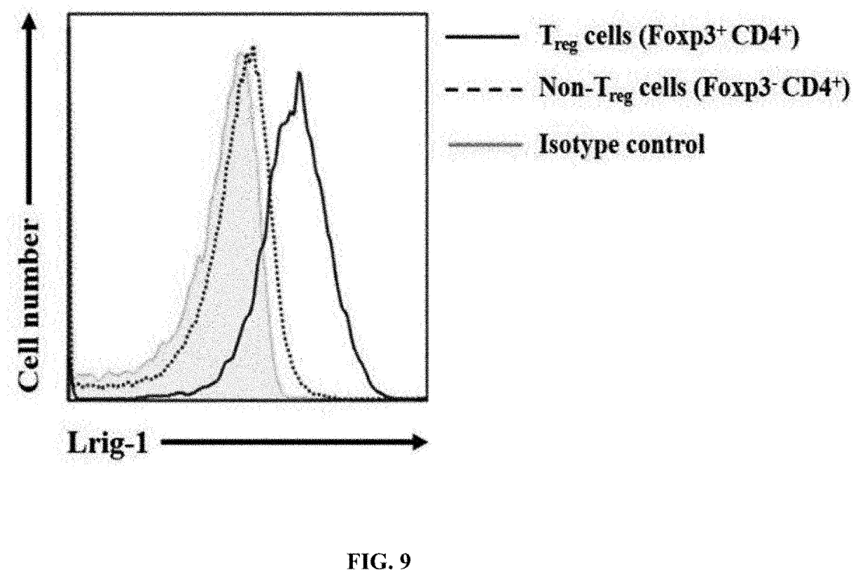

Using FOXP3-RFP-knocked-in mice, the FOXP3-RFP obtained by coupling red fluorescence protein (RFP) to FOXP3 promoter, a transcription factor specific for regulatory T cells, CD4⁺ T cells were isolated using magnet-activated cell sorting (MACS), through CD4 beads, from the spleen of the mice. Subsequently, using RFP protein, regulatory T (CD4⁺RFP⁺ T) cells and non-regulatory T (CD4⁺RFP⁻ T) cells were obtained by performing isolation through a fluorescence-activated cell sorter (FACS). The respective cells were stained with the purchased Lrig-1 antibody and a negative control was stained with an isotype-matched control antibody, to measure an expression level of Lrig-1 with the fluorescence-activated cell sorter. The results are illustrated in FIG. 9.

As illustrated in FIG. 9, the non-regulatory T cells indicated by a dotted line showed almost the same expression level of Lrig-1 as the negative control, whereas there were a large number of cells with high expression level of Lrig-1 in the regulatory T cells.

From the above results, it can be seen that the Lrig-1 protein according to the present invention is specifically expressed in regulatory T cells.

### Example 5 Identification of Specific Expression of Lrig-1 Protein on Surface of Regulatory T Cells

From the viewpoint that in order to be a target of cell therapy, the Lrig-1 protein must be expressed on the surface of regulatory T cells, which in turn allows a more effective target therapy, it was identified whether the Lrig-1 protein is expressed on the surface of the regulatory T cells.

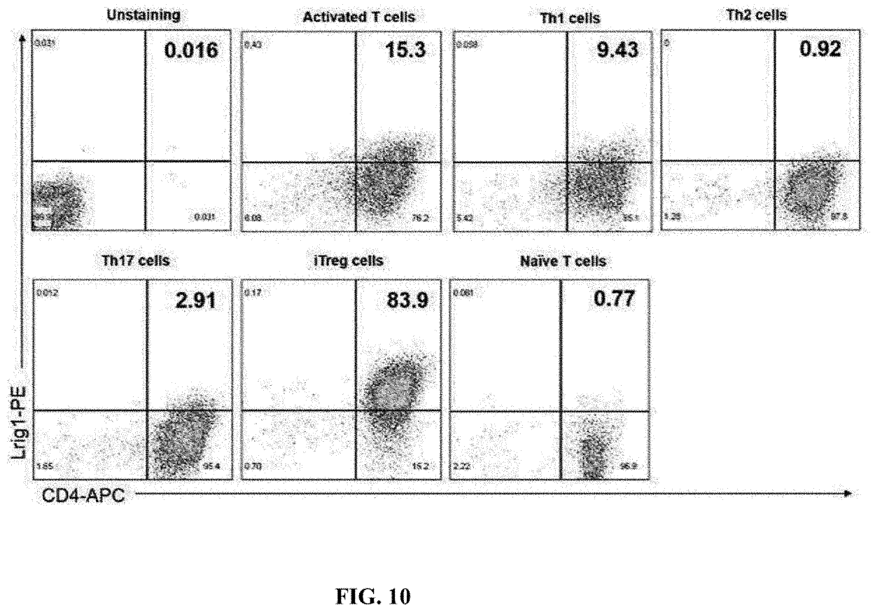

The respective differentiated T cell subsets of Preparation Example 1 were stained with anti-CD4-APC and anti-Lrig-1-PE antibodies, and expression levels of Lrig-1 were measured at the respective cell surfaces using a fluorescence-activated cell sorter (FACS). The results are illustrated in FIG. 10.

As illustrated in FIG. 10, Lrig-1 was expressed in an amount of 0.77 to 15.3 in activated T cells, Th1 cells, Th2 cells, Th17 cells, and naive T cells, whereas Lrig-1 was expressed as high as 83.9 in differentiation-induced T cells (iTreg cells).

From the above results, it can be seen that the Lrig-1 protein according to the present invention is not only specifically expressed in regulatory T (Treg) cells, but also is, in particular, expressed at a higher level on the surface of the Treg cells.

### Example 6 Evaluation of Binding Capacity of Antibody According to Present Invention to Lrig-1 Protein

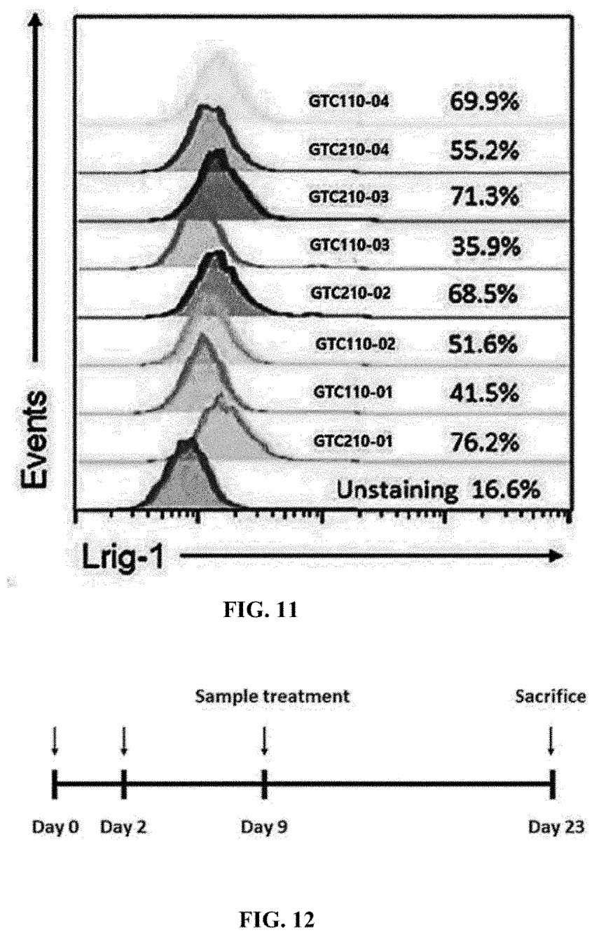

In order to identify whether the monoclonal antibodies according to the present invention produced in Production Examples 1 to 8 well recognize Lrig-1, each of the antibodies of Production Examples 1 to 8 was bound to L cells that stably express Lrig-1. Then, a secondary antibody which is conjugated with eFlour 670 and is capable of recognizing the mouse antibodies was added thereto, and then binding capacity of the monoclonal antibodies to the Lrig-1 protein was analyzed using FACS. The results are illustrated in FIG. 11.

As illustrated in FIG. 11, it was found that all Lrig-1 protein-specific monoclonal antibodies (GTC110-04 and GTC210-01) according to the present invention effectively recognize and bind to the Lrig-1 protein present on the surface of L cells.

### Example 71 Evaluation of Therapeutic Ability of Antibody According to Present Invention Against Multiple Sclerosis

### 1. Production of Mouse Model for Multiple Sclerosis

In order to produce a mouse model for multiple sclerosis, an experiment was performed according to the experimental design illustrated in FIG. 12. On day 0, 7-week-old C57BL/6 mice were subcutaneously injected with MOG peptide and intraperitoneally injected with Mycobacterium tuberculosis toxin. On day 2, the mice were injected once more with the Mycobacterium tuberculosis toxin to enhance their immune system. On day 9, the mice were subjected to administration of the GTC210-01 antibody produced in Production Example 1, and subjected to administration of an anti-IL-17a antibody as a positive control.

### 2. Evaluation of Therapeutic Effect on Multiple Sclerosis

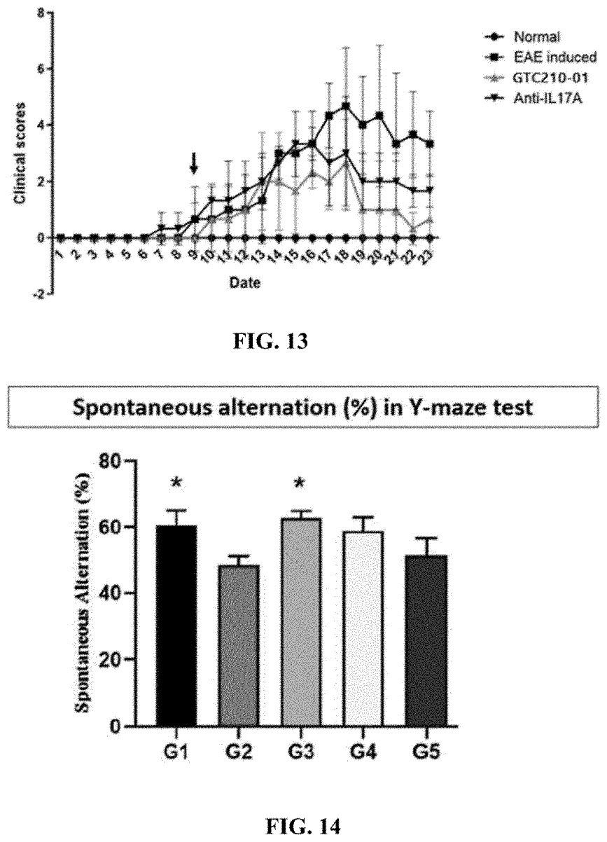

Starting from day 7, a therapeutic effect on multiple sclerosis in the mice was evaluated by calculating a clinical score according to the following evaluation criteria, and the results are illustrated in FIG. 13.

### Evaluation Criteria

0 points: Normal movement with no symptoms

1 point: Part of the tip of the tail is paralyzed and hangs down

2 points: The entire tail is paralyzed and is limp.

3 points: One of the hindlimbs is partially paralyzed but is able to respond to stimuli

4 points: One of the hindlimbs is completely paralyzed and does not respond to stimuli, causing the limb to drag when moving

5 points: Both hindlimbs are paralyzed and body dragging is made with the forelimbs

6 points: It is difficult to make body dragging with the forelimbs and the forelimbs still respond to stimuli.

7 points: The mouse is unable to move but one forelimb responds to stimuli.

8 points: The mouse is unable to move and both forelimbs do not respond to stimuli.

9 points: The mouse is unable to move and has irregular breathing

10 points: The mouse dies

As a result, it was found that in a case where the model with induced multiple sclerosis was administered with the antibody according to the present invention, multiple sclerosis was remarkably treated. In particular, it was found that on days 22 and 23, the model was recovered to a level similar to that of normal mice.

### Example 8 Evaluation of Therapeutic Ability of Antibody According to Present Invention Against Alzheimer's Disease

In order to evaluate the therapeutic ability of the antibody according to the present invention against Alzheimer's disease, groups were designed as shown in Table 4. Specifically, 7-month-old 5xFAD mice with induced Alzheimer's disease were intravenously injected with GTC110-04 antibody or GTC210-01 antibody in an amount of 10 mpk each for 1 month. As a positive control, 8-month-old mice with Alzheimer's disease were subcutaneously injected with glatiramer acetate (GA, Copaxone®) in an amount of 100 ug for 3 weeks.

TABLE 4

| | | | | Aministration | | |
|---|---|---|---|---|---|---|
| Group | | | n/group | route | Dose | |
| G1 | WT | Control | 14 | — | — | |
| G2 | 5xFAD | Control | 13 | — | — | |
| G3 | | GTC110-04 antibody | 11 | IV | 10 | mpk |
| G4 | | GTC210-01 antibody | 11 | IV | 10 | mpk |
| G5 | | GA (positive control) | 11 | SC | 100 | ug |

### 1. Y-Maze Test

For the Y-maze test, a Y-shaped maze was used which had three identical arms having a length of 40 cm (with a 15-cm high wall) at an angle of 120 degrees from each other. This experiment was a behavioral experiment using the rodent's instinctive exploring habit and was based on the fact that there is a high possibility of exploring a new area. The higher the degree that the test animal remembered the arm it had just explored and tried not to enter the same arm, the higher the animal's memory level. An exploring time of 8 minutes was given to each individual, and the final results were expressed as spontaneous alteration (%) values in FIG. 14. The spontaneous alteration (%) value was calculated by Expression 1. Here, behavioral patterns were analyzed using SMART VIDEO TRACKING Software (Panlab, USA).

Spontaneous alternation (%)=The number of triplet/(total arm entry-2) [Expression 1]

As a result, in this experiment for measuring the relative frequency at which the test animal perceives the surrounding clues and sequentially enters the maze, it was found that in a case where the mice with induced Alzheimer's disease were administered with the antibodies (GTC210-01 and GTC110-04 antibodies) according to the present invention, the mice had a spontaneous alternation value at a level similar to that of a normal control.

### 2. Novel Object Recognition Test

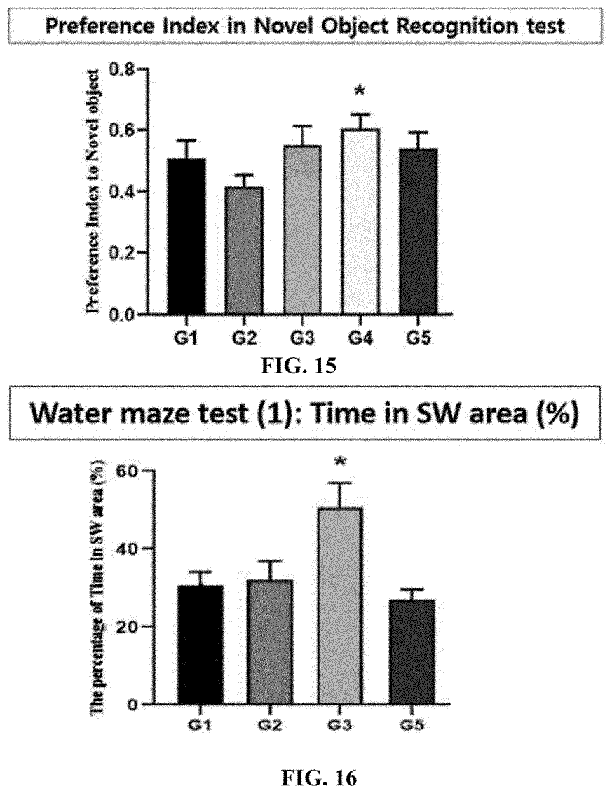

The novel object recognition test was performed to evaluate memory using two different objects in a 40 cm×40 cm acrylic cage. After causing the test animal to acclimatize to the inside of the acrylic cage, two objects were placed at certain locations and the animal was allowed to perceive the objects freely. Then, the time for exploring each object was measured. 24-hour delay was given to each individual, and only one object was changed to another at the same location. Here, in a case where the animal perceives the changed object as a novel object and spends a longer exploring time, it may be determined that the animal has better memory. In a case where the animal does not remember the objects that it explored 24 hours before, the animal fails to distinguish between the novel object and the old object, and thus there is a possibility that the animal explores equally both objects. The animal was allowed to explore freely for a total of 10 minutes, and the result was expressed as a preference index (which equals novel object exploring time/total exploring time) in FIG. 15. Here, the analysis was performed using SMART VIDEO TRACKING Software (Panlab, USA).

As a result, regarding the preference index for the novel object, the normal control (G1) was measured to be 0.51±0.06, and the G2 group (vehicle) was measured to be 0.42 35 0.04. Thus, it was observed that the group with induced Alzheimer's disease (G2) has a low preference index as compared with the normal control (G1). On the other hand, it was found that the group having received the antibodies (GTC210-01 and GTC110-04 antibodies) according to the present invention had a higher preference index than the normal control.

### 3. Water Maze Test

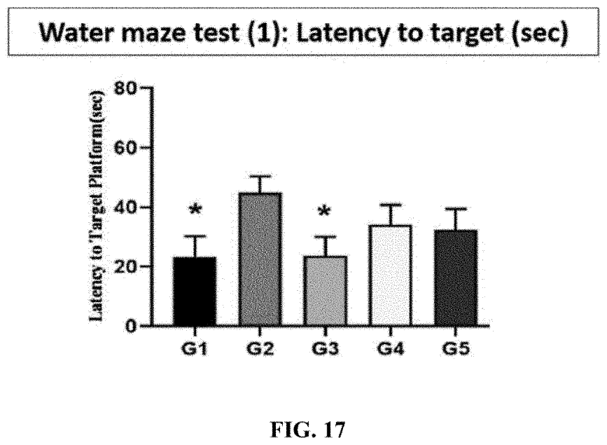

The water maze test was conducted based on the method devised by Morris. A stainless steel pool (90 cm in diameter, 50 cm in height) was filled with water (22±1° C.) so that the height of the water surface was 30 cm. A hidden platform (5 cm in diameter) was placed 1 cm below the water surface. On day 1 of evaluation, 3 to 4 training sessions were allowed. A total swimming time per individual was set to 60 seconds; and the individual, who found the platform within 60 seconds, was allowed to stay on the platform for 10 seconds to induce memory. The individual, who was unable to find the platform even after 60 seconds, was manually guided to the platform and allowed to stay on the platform for 10 seconds, wherein the time to escape was set to 60 seconds. On day 6 after the training, a probe test was conducted to measure the value (percentage of time in SW area, %), which is spatial perception, obtained by calculating the time spent in the quadrant where the platform was as a percentage of the total swimming time, and the time (latency to target, sec) taken to reach the location where the platform was. The results are illustrated in FIGS. 16 and 17. Here, the analysis was performed using SMART VIDEO TRACKING Software (Panlab, USA). In interpreting the results, the individual included in the exclusion criteria was set as an individual who moves around a specific area without exploration through swimming.

As a result, it was found that the group having received the antibodies (GTC210-01 and GTC110-04 antibodies) according to the present invention had a remarkably increased value (percentage of time in SW area, %), obtained by calculating the time spent in the quadrant where the platform was as a percentage of the total swimming time, and had a remarkably decreased time (latency to target, sec) taken to reach the location where the platform was, which was a level similar to that of the normal control.

Through these experiments, it was found that the antibody according to the present invention had a prophylactic, ameliorating, or therapeutic effect on brain and nervous system diseases.

Although the present invention has been described in detail above, the scope of the present invention is not limited thereto. It will be obvious to those skilled in the art that various modifications and changes can be made without departing from the technical spirit of the present invention described in the claims.

### INDUSTRIAL APPLICABILITY

The present invention relates to a composition for preventing or treating a brain and nervous system disease.

| | |
|---|---|
| Sequence List Free Text | |
| SEQ ID NO 1: | |
| GPRAPCAAAC TCAGDSLDCG GRGLAALPGD LPSWTRSLNL SYNKLSEIDP AGFEDLPNLQ | 60 |
| EVYLNNNELT AVPSLGAASS HVVSLFLQHN KIRSVEGSQL KAYLSLEVLD LSLNNITEVR | 120 |
| NTCFPHGPPI KELNLAGNRI GTLELGAFDG LSRSLLTLRL SKNRITQLPV RAFKLPRLTQ | 180 |
| LDLNRNRIRL IEGLTFQGLN SLEVLKLQRN NISKLTDGAF WGLSKMHVLH LEYNSLVEVN | 240 |
| SGSLYGLTAL HQLHLSNNSI ARIHRKGWSF CQKLHELVLS FNNLTRLDEE SLAELSSLSV | 300 |
| LRLSHNSISH IAEGAFKGLR SLRVLDLDHN EISGTIEDTS GAFSGLDSLS KLTLFGNKIK | 360 |
| SVAKRAFSGL EGLEHLNLGG NAIRSVQFDA FVKMKNLKEL HISSDSFLCD CQLKWLPPWL | 420 |
| IGRMLQAFVT ATCAHPESLK GQSIFSVPPE SFVCDDFLKP QIITQPETTM AMVGKDIRFT | 480 |
| CSAASSSSSP MTFAWKKDNE VLTNADMENF VHVHAQDGEV MEYTTILHLR QVTFGHEGRY | 540 |
| QCVITNHFGS TYSHKARLTV NVLPSFTKTP HDITIRTTTV ARLECAATGH PNPQIAWQKD | 600 |
| GGTDFPAARE RRMHVMPDDD VFFITDVKID DAGVYSCTAQ NSAGSISANA TLTVLETPSL | 660 |
| VVPLEDRVVS VGETVALQCK ATGNPPPRIT WFKGDRPLSL TERHHLTPDN QLLVVQNVVA | 720 |
| EDAGRYTCEM SNTLGTERAH SQLSVLPAAG CRKDGTTVG | |
| SEQ ID NO 2: | |
| ggcccgcggg cgccctgcgc ggccgcctgc acttgcgctg gggactcgct ggactgcggt | 60 |
| gggcgcgggc tggctgcgtt gcccggggac ctgccctcct ggacgcggag cctaaacctg | 120 |
| agttacaaca aactctctga gattgaccct gctggttttg aggacttgcc gaacctacag | 180 |
| gaagtgtacc tcaataataa tgagttgaca gcggtaccat ccctgggcgc tgcttcatca | 240 |
| catgtcgtct ctctctttct gcagcacaac aagattcgca gcgtggaggg gagccagctg | 300 |
| aaggcctacc tttccttaga agtgttagat ctgagtttga acaacatcac ggaagtgcgg | 360 |
| aacacctgct ttccacacgg accgcctata aaggagctca acctggcagg caatcggatt | 420 |
| ggcaccctgg agttgggagc atttgatggt ctgtcacggt cgctgctaac tcttcgcctg | 480 |
| agcaaaaaca ggatcaccca gcttcctgta agagcattca agctacccag gctgacacaa | 540 |
| ctggacctca atcggaacag gattcggctg atagagggcc tcaccttcca ggggctcaac | 600 |
| agcttggagg tgctgaagct tcagcgaaac aacatcagca aactgacaga tggggccttc | 660 |
| tggggactgt ccaagatgca tgtgctgcac ctggagtaca acagcctggt agaagtgaac | 720 |
| agcggctcgc tctacggcct cacggccctg catcagctcc acctcagcaa caattccatc | 780 |
| gctcgcattc accgcaaggg ctggagcttc tgccagaagc tgcatgagtt ggtcctgtcc | 840 |
| ttcaacaacc tgacacggct ggacgaggag agcctggccg agctgagcag cctgagtgtc | 900 |
| ctgcgtctca gccacaattc catcagccac attgcggagg gtgccttcaa gggactcagg | 960 |
| agcctgcgag tcttggatct ggaccataac gagatttcgg gcacaataga ggacacgagc | 1020 |
| ggcgccttct cagggctcga cagcctcagc aagctgactc tgtttggaaa caagatcaag | 1080 |
| tctgtggcta agagagcatt ctcggggctg gaaggcctgg agcacctgaa ccttggaggg | 1140 |
| aatgcgatca gatctgtcca gtttgatgcc tttgtgaaga tgaagaatct taaagagctc | 1200 |
| catatcagca gcgacagctt cctgtgtgac tgccagctga agtggctgcc cccgtggcta | 1260 |
| attggcagga tgctgcaggc ctttgtgaca gccacctgtg cccacccaga atcactgaag | 1320 |
| ggtcagagca ttttctctgt gccaccagag agtttcgtgt gcgatgactt cctgaagcca | 1380 |
| cagatcatca cccagccaga aaccaccatg gctatggtgg gcaaggacat ccggtttaca | 1440 |
| tgctcagcag ccagcagcag cagctccccc atgacctttg cctggaagaa agacaatgaa | 1500 |
| gtcctgacca atgcagacat ggagaacttt gtccacgtcc acgcgcagga cggggaagtg | 1560 |
| atggagtaca ccaccatcct gcacctccgt caggtcactt tcgggcacga gggccgctac | 1620 |
| caatgtgtca tcaccaacca ctttggctcc acctattcac ataaggccag gctcaccgtg | 1680 |
| aatgtgttgc catcattcac caaaacgccc cacgacataa ccatccggac caccaccgtg | 1740 |
| gcccgcctcg aatgtgctgc cacaggtcac ccaaaccctc agattgcctg gcagaaggat | 1800 |
| ggaggcacgg atttccccgc tgcccgtgag cgacgcatgc atgtcatgcc ggatgacgac | 1860 |
| gtgtttttca tcactgatgt gaaaatagat gacgcagggg tttacagctg tactgctcag | 1920 |
| aactcagccg gttctatttc agctaatgcc accctgactg tcctagagac cccatccttg | 1980 |
| gtggtcccct tggaagaccg tgtggtatct gtgggagaaa cagtggccct ccaatgcaaa | 2040 |
| gccacgggga accctccgcc ccgcatcacc tggttcaagg gggaccgccc gctgagcctc | 2100 |
| actgagcggc accacctgac ccctgacaac cagctcctgg tggttcagaa cgtggtggca | 2160 |
| gaggatgcgg gccgatatac ctgtgagatg tccaacaccc tgggcacgga gcgagctcac | 2220 |
| agccagctga gcgtcctgcc cgcagcaggc tgcaggaagg atgggaccac ggtaggcatc | 2280 |
| ttcaccattg ctgtcgtgag cagcatcgtc ctgacgtcac tggtctgggt gtgcatcatc | 2340 |
| taccagacca ggaagaagag tgaagagtac agtgtcacca acacagatga aaccgtc | |
| SEQ ID NO 3: | |
| QAGPRAPCAA ACTCAGDSLD CSGRGLATLP RDLPSWTRSL NLSYNRLSEI DSAAFEDLTN | 60 |
| LQEVYLNSNE LTAIPSLGAA SIGVVSLFLQ HNKILSVDGS QLKSYLSLEV LDLSSNNITE | 120 |
| IRSSCFPNGL RIRELNLASN RISILESGAF DGLSRSLLTL RLSKNRITQL PVKAFKLPRL | 180 |
| TQLDLNRNRI RLIEGLTFQG LDSLEVLRLQ RNNISRLTDG AFWGLSKMHV LHLEYNSLVE | 240 |
| VNSGSLYGLT ALHQLHLSNN SISRIQRDGW SFCQKLHELI LSFNNLTRLD EESLAELSSL | 300 |
| SILRLSHNAI SHIAEGAFKG LKSLRVLDLD HNEISGTIED TSGAFTGLDN LSKLTLFGNK | 360 |
| IKSVAKRAFS GLESLEHLNL GENAIRSVQF DAFAKMKNLK ELYISSESFL CDCQLKWLPP | 420 |
| WLMGRMLQAF VTATCAHPES LKGQSIFSVL PDSFVCDDFP KPQIITQPET TMAVVGKDIR | 480 |
| FTCSAASSSS SPMTFAWKKD NEVLANADME NFAHVRAQDG EVMEYTTILH LRHVTFGHEG | 540 |
| RYQCIITNHF GSTYSHKARL TVNVLPSFTK IPHDIAIRTG TTARLECAAT GHPNPQIAWQ | 600 |
| KDGGTDFPAA RERRMHVMPD DDVFFITDVK IDDMGVYSCT AQNSAGSVSA NATLTVLETP | 660 |
| SLAVPLEDRV VTVGETVAFQ CKATGSPTPR ITWLKGGRPL SLTERHHFTP GNQLLVVQNV | 720 |
| MIDDAGRYTC EMSNPLGTER AHSQLSILPT PGCRKDGTTV G | |
| SEQ ID NO 4: | |
| caggctggcc cgcgggcccc ctgcgcggcc gcctgcactt gcgccgggga ctcgctggac | 60 |
| tgcagtgggc gcgggctggc gacgctgccc cgggacctgc cctcctggac gcgcagccta | 120 |
| aacctgagtt ataacagact ctccgagatc gactctgctg cttttgagga cttgacgaat | 180 |
| ctgcaggaag tgtacctcaa cagcaatgag ctgacagcca taccatcact gggcgctgct | 240 |
| tccataggag ttgtctctct ctttttgcag cacaacaaga tccttagtgt ggatgggagc | 300 |
| cagctgaagt cgtacctgtc cttggaagtg ctggatctga gttccaacaa catcacggaa | 360 |
| attcggagct cctgtttccc gaacggcctg cgtataaggg aactcaactt ggcgagcaac | 420 |
| cgcatcagca tcctggagtc tggagcattt gatggtctgt cgcggtcact gctgactctc | 480 |
| cgtctgagca aaaacaggat cacccagctt cctgtgaaag cgttcaagct acccaggctg | 540 |
| acacaactag acctgaatcg gaatcggatt cggctgattg aaggcctcac gttccagggg | 600 |
| ctcgacagct tagaggtgct gaggcttcag aggaacaaca tcagcaggct gacggacggg | 660 |
| gccttctggg ggctgtctaa gatgcacgtg ctgcacctgg agtacaacag tctggtggaa | 720 |
| gtgaacagtg gctccctcta tggcctcaca gccctgcacc agctgcacct cagcaacaac | 780 |
| tccatctctc gaattcagcg tgatggctgg agcttctgcc aaaagctgca tgagttgatt | 840 |
| ctgtccttca acaacctcac gcggctggat gaggagagtc tagcggagtt gagcagcctc | 900 |
| agtatcctgc gcctcagtca caacgccatc agtcacattg ctgaaggcgc cttcaaggga | 960 |
| ctcaagagtc tgcgggtctt ggacctggac cataacgaga tctcgggtac aatcgaggat | 1020 |
| accagtggtg cctttacggg gcttgacaac ctcagcaagc tgactctgtt tggaaacaag | 1080 |
| atcaaatctg tggctaagag agccttctcg ggcctggaaa gcctggaaca cctgaacctt | 1140 |
| ggagagaatg caatcaggtc tgtccagttt gatgcctttg caaagatgaa gaaccttaaa | 1200 |
| gagctctaca tcagcagtga gagcttcctg tgtgactgcc agctcaagtg gctgccccca | 1260 |
| tggctaatgg gtaggatgct gcaggccttt gtgacagcca cctgtgccca tccagagtcg | 1320 |
| ctgaagggcc agagcatttt ctcagtgctg ccagacagct ttgtgtgtga tgactttcca | 1380 |
| aagccacaga tcatcaccca gcctgagacg accatggctg tggtgggcaa ggacatccgt | 1440 |
| ttcacatgct ccgcagccag cagcagcagc tcaccaatga ccttcgcctg gaagaaggac | 1500 |
| aatgaggtcc tggccaatgc agacatggag aactttgccc acgtccgtgc acaggacggc | 1560 |
| gaagtgatgg agtataccac tatcctgcac ctccgtcacg tcacctttgg gcacgagggc | 1620 |
| cgctaccagt gtatcatcac aaaccacttt ggctccacat actcccacaa agccaggctc | 1680 |
| actgtgaatg tgttgccatc attcactaaa ataccccatg acattgccat ccggactggc | 1740 |
| accacagccc gcctcgagtg tgctgccacg ggccacccta accctcagat tgcctggcag | 1800 |
| aaggatggag gcaccgattt cccggcagct cgtgagcgac gcatgcatgt tatgccagac | 1860 |
| gatgatgtgt tcttcatcac tgatgtgaaa atagacgaca tgggggtcta cagctgcact | 1920 |
| gcccagaact cggcaggctc ggtttcagcc aacgctaccc tcacagtctt agaaactcca | 1980 |
| tccttggcag tgcctctgga agaccgtgtg gtaactgtgg gagaaacagt ggccttccag | 2040 |
| tgcaaagcaa ccgggagccc cacaccacgc atcacctggc ttaagggagg tcgcccattg | 2100 |
| agcctcacag agcgccacca tttcactcca ggcaaccagc tgctggttgt tcagaatgtg | 2160 |
| atgatagacg atgcagggcg gtatacctgt gagatgtcta atcccctggg cactgagcga | 2220 |
| gcacatagcc agctgagcat tttacctacc cctggctgcc ggaaggatgg gaccaccgta | 2280 |
| ggc | |
| SEQ ID NO 5: | |
| GFTFSGYD | |
| SEQ ID NO 6: | |
| LIYPDSGNK | |
| SEQ ID NO 7: | |
| ARDAGLSWAG AFDY | |
| SEQ ID NO 8: | |
| SSNIGSNY | |
| SEQ ID NO 9: | |
| SDS | |
| SEQ ID NO 10: | |
| GSWDYSLSAY V | |
| SEQ ID NO 11: | |
| EVQLLESGGG LVQPGGSLRL SCAASGFTFS GYDMSWVRQA PGKGLEWVSL IYPDSGNKYY | 60 |
| ADSVKGRFTI SRDNSKNTLY LQMNSLRAED TAVYYCARDA GLSWAGAFDY WGQGTLVTVS | 120 |
| S | |
| SEQ ID NO 12: | |
| QSVLTQPPSA SGTPGQRVTI SCSGSSSNIG SNYVTWYQQL PGTAPKLLIY SDSHRPSGVP | 60 |
| DRFSGSKSGT SASLAISGLQ SEDEADYYCG SWDYSLSAYV FGGGTKLTVL | |
| SEQ ID NO 13: | |
| GFTFSNYA | |
| SEQ ID NO 14: | |
| VISHGGGST | |
| SEQ ID NO 15: | |
| ARVISNCHLG VCYYSNGMDV | |
| SEQ ID NO 16: | |
| SSNIGNND | |
| SEQ ID NO 17: | |
| SDS | |
| SEQ ID NO 18: | |
| GTWDYSLSGY V | |
| SEQ ID NO 19: | |
| EVQLLESGGG LVQPGGSLRL SCAASGFTFS NYAMSWVRQA PGKGLEWVSV ISHGGGSTYY | 60 |
| ADSVKGRFTI SRDNSKNTLY LQMNSLRAED TAVYYCARVI SNCHLGVCYY SNGMDVWGQG | 120 |
| TLVTVSS | |
| SEQ ID NO 20: | |
| QSVLTQPPSA SGTPGQRVTI SCSGSSSNIG NNDVYWYQQL PGTAPKLLIY SDSQRPSGVP | 60 |
| DRFSGSKSGT SASLAISGLR SEDEADYYCG TWDYSLSGYV FGGGTKLTVL | |
| SEQ ID NO 21: | |
| TTAPSVYPLA PVCGDTTGSS VTLGCLVKGY FPEPVTLTWN SGSLSSGVHT FPAVLQSDLY | 60 |
| TLSSSVTVTS STWPSQSITC NVAHPASSTK VDKKIEPRGP TIKPCPPCKC PAPNLLGGPS | 120 |
| VFIFPPKIKD VLMISLSPIV TCVVVDVSED DPDVQISWFV NNVEVHTAQT QTHREDYNST | 180 |
| LRVVSALPIQ HQDWMSGKEF KCKVNNKDLP APIERTISKP KGSVRAPQVY VLPPPEEEMT | 240 |
| KKQVTLTCMV TDFMPEDIYV EWTNNGKTEL NYKNTEPVLD SDGSYFMYSK LRVEKKNWVE | 300 |
| RNSYSCSVVH EGLHNHHTTK SFSRTPGK | |
| SEQ ID NO 22: | |
| RTVAAPTVSI FPPSSEQLTS GGASVVCFLN NFYPKDINVK WKIDGSERQN GVLNSWTDQD | 60 |
| SKDSTYSMSS TLTLTKDEYE RHNSYTCEAT HKTSTSPIVK SFNRNEC | |
| SEQ ID NO 23: | |
| ASTKGPSVFP LAPSSKSTSG GTAALGCLVK DYFPEPVTVS WNSGALTSGV HTFPAVLQSS | 60 |
| GLYSLSSVVT VPSSSLGTQT YICNVNHKPS NTKVDKKVEP KSCDKTHTCP PCPAPELLGG | 120 |
| PSVFLFPPKP KDTLMISRTP EVTCVVVDVS HEDPEVKFNW YVDGVEVHNA KTKPREEQYN | 180 |
| STYRVVSVLT VLHQDWLNGK EYKCKVSNKA LPAPIEKTIS KAKGQPREPQ VYTLPPSRDE | 240 |
| LTKNQVSLTC LVKGFYPSDI AVEWESNGOP ENNYKTTPPV LDSDGSFFLY SKLTVDKSRW | 300 |
| QQGNVFSCSV MHEALHNHYT QKSLSLSPGK | |
| SEQ ID NO 24: | |
| ASTKGPSVFP LAPSSKSTSG GTAALGCLVK DYFPEPVTVS WNSGALTSGV HTFPAVLQSS | 60 |
| GLYSLSSVVT VPSSSLGTQT YICNVNHKPS NTKVDKKVEP KSCDKTHTCP PCPAPELLGG | 120 |
| PSVFLFPPKP KDTLMISRTP EVTCVVVDVS HEDPEVKFNW YVDGVEVHNA KTKPREEQYN | 180 |
| STYRVVSVLT VLHQDWLNGK EYKCKVSNKA LPAPIEKTIS KAKGQPREPQ VYTLPPSREE | 240 |
| MTKNQVSLTC LVKGFYPSDI AVEWESNGQP ENNYKTTPPV LDSDGSFFLY SKLTVDKSRW | 300 |
| QQGNVFSCSV MHEALHNHYT QKSLSLSPGK | |
| SEQ ID NO 25: | |
| RTVAAPSVFI FPPSDEQLKS GTASVVCLLN NFYPREAKVQ WKVDNALQSG NSQESVTEQD | 60 |
| SKDSTYSLSS TLTLSKADYE KHKVYACEVT HQGLSSPVTK SFNRGEC | |
| SEQ ID NO 26: | |
| ASTKGPSVFP LAPCSRSTSE STAALGCLVK DYFPEPVTVS WNSGALTSGV HTFPAVLQSS | 60 |
| GLYSLSSVVT VPSSNFGTQT YTCNVDHKPS NTKVDKTVER KCCVECPPCP APPVAGPSVF | 120 |
| LFPPKPKDTL MISRTPEVTC VVVDVSHEDP EVQFNWYVDG VEVHNAKTKP REEQFNSTFR | 180 |
| VVSVLTVVHQ DWLNGKEYKC KVSNKGLPAP IEKTISKTKG QPREPQVYTL PPSREEMTKN | 240 |
| QVSLTCLVKG FYPSDISVEW ESNGQPENNY KTTPPMLDSD GSFFLYSKLT VDKSRWQQGN | 300 |
| VFSCSVMHEA LHNHYTQKSL SLSPGK | |
| SEQ ID NO 27: | |
| ASTKGPSVFP LAPCSRSTSG GTAALGCLVK DYFPEPVTVS WNSGALTSGV HTFPAVLQSS | 60 |
| GLYSLSSVVT VPSSSLGTQT YTCNVNHKPS NTKVDKRVEL KTPLGDTTHT CPRCPEPKSC | 120 |
| DTPPPCPRCP EPKSCDTPPP CPRCPEPKSC DTPPPCPRCP APELLGGPSV FLFPPKPKDT | 180 |
| LMISRTPEVT CVVVDVSHED PEVQFKWYVD GVEVHNAKTK PREEQYNSTF RVVSVLTVLH | 240 |
| QDWLNGKEYK CKVSNKALPA PIEKTISKTK GQPREPQVYT LPPSREEMTK NQVSLTCLVK | 300 |
| GFYPSDIAVE WESSGQPENN YNTTPPMLDS DGSFFLYSKL TVDKSRWQQG NIFSCSVMHE | 360 |
| ALHNRFTQKS LSLSPGK | |
| SEQ ID NO 28: | |
| ASTKGPSVFP LAPCSRSTSE STAALGCLVK DYFPEPVTVS WNSGALTSGV HTFPAVLQSS | 60 |
| GLYSLSSVVT VPSSSLGTKT YTCNVDHKPS NTKVDKRVES KYGPPCPSCP APEFLGGPSV | 120 |
| FLFPPKPKDT LMISRTPEVT CVVVDVSQED PEVQFNWYVD GVEVHNAKTK PREEQFNSTY | 180 |
| RVVSVLTVLH QDWLNGKEYK CKVSNKGLPS SIEKTISKAK GQPREPQVYT LPPSQEEMTK | 240 |
| NQVSLTCLVK GFYPSDIAVE WESNGQPENN YKTTPPVLDS DGSFFLYSRL TVDKSRWQEG | 300 |
| NVFSCSVMHE ALHNHYTQKS LSLSLGK | |
| SEQ ID NO 29: | |
| RNTGRGGEEK KKEKEKEEQE ERETKTPECP SHTQPLGVFL FPPKPKDTLM ISRTPEVTCV | 60 |
| VVDVSQEDPE VQFNWYVDGV EVHNAKTKPR EEQFNSTYRV VSVLTVLHQD WLNGKEYKCK | 120 |
| VSNKGLPSSI EKTISKAKGQ PREPQVYTLP PSQEEMTKNQ VSLTCLVKGF YPSDIAVEWE | 180 |
| SNGQPENNYK TTPPVLDSDG SFFLYSRLTV DKSRWQEGNV FSCSVMHEAL HNHYTQKSLS | 240 |
| LSLGK | |
| SEQ ID NO 30: | |
| EVQLLESGGG LVQPGGSLRL SCAASGFTFS GYDMSWVRQA PGKGLEWVSL IYPDSGNKYY | 60 |
| ADSVKGRFTI SRDNSKNTLY LQMNSLRAED TAVYYCARDA GLSWAGAFDY WGQGTLVTVS | 120 |
| SASTTAPSVY PLAPVCGDTT GSSVTLGCLV KGYFPEPVTL TWNSGSLSSG VHTFPAVLQS | 180 |
| DLYTLSSSVT VTSSTWPSQS ITCNVAHPAS STKVDKKIEP RGPTIKPCPP CKCPAPNLLG | 240 |
| GPSVFIFPPK IKDVLMISLS PIVTCVVVDV SEDDPDVQIS WFVNNVEVHT AQTQTHREDY | 300 |
| NSTLRVVSAL PIQHQDWMSG KEFKCKVNNK DLPAPIERTI SKPKGSVRAP QVYVLPPPEE | 360 |
| EMTKKQVTLT CMVTDFMPED IYVEWTNNGK TELNYKNTEP VLDSDGSYFM YSKLRVEKKN | 420 |
| WVERNSYSCS VVHEGLHNHH TTKSFSRTPG K | |
| SEQ ID NO 31: | |
| QSVLTQPPSA SGTPGQRVTI SCSGSSSNIG SNYVTWYQQL PGTAPKLLIY SDSHRPSGVP | 60 |
| DRFSGSKSGT SASLAISGLQ SEDEADYYCG SWDYSLSAYV FGGGTKLTVL RTVAAPTVSI | 120 |
| FPPSSEQLTS GGASVVCFLN NFYPKDINVK WKIDGSERQN GVLNSWTDQD SKDSTYSMSS | 180 |
| TLTLTKDEYE RHNSYTCEAT HKTSTSPIVK SFNRNEC | |
| SEQ ID NO 32: | |
| EVQLLESGGG LVQPGGSLRL SCAASGFTFS NYAMSWVRQA PGKGLEWVSV ISHGGGSTYY | 60 |
| ADSVKGRFTI SRDNSKNTLY LQMNSLRAED TAVYYCARVI SNCHLGVCYY SNGMDVWGQG | 120 |
| TLVTVSSAST TAPSVYPLAP VCGDTTGSSV TLGCLVKGYF PEPVTLTWNS GSLSSGVHTF | 180 |
| PAVLQSDLYT LSSSVTVTSS TWPSQSITCN VAHPASSTKV DKKIEPRGPT IKPCPPCKCP | 240 |
| APNLLGGPSV FIFPPKIKDV LMISLSPIVT CVVVDVSEDD PDVQISWFVN NVEVHTAQTQ | 300 |
| THREDYNSTL RVVSALPIQH QDWMSGKEFK CKVNNKDLPA PIERTISKPK GSVRAPQVYV | 360 |
| LPPPEEEMTK KQVTLTCMVT DFMPEDIYVE WTNNGKTELN YKNTEPVLDS DGSYFMYSKL | 420 |
| RVEKKNWVER NSYSCSVVHE GLHNHHTTKS FSRTPGK | |
| SEQ ID NO 33: | |
| QSVLTQPPSA SGTPGQRVTI SCSGSSSNIG NNDVYWYQQL PGTAPKLLIY SDSQRPSGVP | 60 |
| DRFSGSKSGT SASLAISGLR SEDEADYYCG TWDYSLSGYV FGGGTKLTVL RTVAAPTVSI | 120 |
| FPPSSEQLTS GGASVVCFLN NFYPKDINVK WKIDGSERQN GVLNSWTDQD SKDSTYSMSS | 180 |
| TLTLTKDEYE RHNSYTCEAT HKTSTSPIVK SFNRNEC | |
| SEQ ID NO 34: | |
| EVQLLESGGG LVQPGGSLRL SCAASGFTFS NYAMSWVRQA PGKGLEWVSV ISHGGGSTYY | 60 |
| ADSVKGRFTI SRDNSKNTLY LQMNSLRAED TAVYYCARVI SNCHLGVCYY SNGMDVWGQG | 120 |
| TLVTVSSAST KGPSVFPLAP SSKSTSGGTA ALGCLVKDYF PEPVTVSWNS GALTSGVHTF | 180 |
| PAVLQSSGLY SLSSVVTVPS SSLGTQTYIC NVNHKPSNTK VDKKVEPKSC DKTHTCPPCP | 240 |
| APELLGGPSV FLFPPKPKDT LMISRTPEVT CVVVDVSHED PEVKFNWYVD GVEVHNAKTK | 300 |
| PREEQYNSTY RVVSVLTVLH QDWLNGKEYK CKVSNKALPA PIEKTISKAK GQPREPQVYT | 360 |
| LPPSREEMTK NQVSLTCLVK GFYPSDIAVE WESNGQPENN YKTTPPVLDS DGSFFLYSKL | 420 |
| TVDKSRWQQG NVFSCSVMHE ALHNHYTQKS LSLSPGKY | |
| SEQ ID NO 35: | |
| QSVLTQPPSA SGTPGQRVTI SCSGSSSNIG NNDVYWYQQL PGTAPKLLIY SDSQRPSGVP | 60 |
| DRFSGSKSGT SASLAISGLR SEDEADYYCG TWDYSLSGYV FGGGTKLTVL RTVAAPSVFI | 120 |
| FPPSDEQLKS GTASVVCLLN NFYPREAKVQ WKVDNALQSG NSQESVTEQD SKDSTYSLSS | 180 |
| TLTLSKADYE KHKVYACEVT HQGLSSPVTK SFNRGEC | |

1-26. (canceled)

27. A method for preventing or treating a nervous system disease in a subject in need thereof, the method comprising administering to the subject a pharmaceutically effective amount of a pharmaceutical composition, wherein the pharmaceutical composition comprises a binding molecule that specifically binds to leucine-rich and immunoglobulin-like domains 1 (Lrig-1) protein present on the surface of regulatory T cells, or a polynucleotide encoding the binding molecule.

28. The method of claim 27, wherein the Lrig-1 protein comprises the amino acid sequence as set forth in SEQ ID NO: 1 or 3.

29. The method of claim 28, wherein the Lrig-1 protein is encoded by a polynucleotide having the nucleotide sequence as set forth in SEQ ID NO: 2 or 4.

30. The method of claim 27, wherein the binding molecule comprises a heavy chain variable region and a light chain variable region,
wherein the heavy chain variable region comprises a heavy chain CDR1 having the amino acid sequence as set forth in SEQ ID NO: 5 or 13, a heavy chain CDR2 having the amino acid sequence as set forth in SEQ ID NO: 6 or 14, and a heavy chain CDR3 having the amino acid sequence as set forth in SEQ ID NO: 7 or 15, and
wherein the light chain variable region comprises a light chain CDR1 having the amino acid sequence as set forth in SEQ ID NO: 8 or 16, a light chain CDR2 having the amino acid sequence as set forth in SEQ ID NO: 9 or 17, and a light chain CDR3 having the amino acid sequence as set forth in SEQ ID NO: 10 or 18.

31. The method of claim 30, wherein the heavy chain variable region comprises a heavy chain CDR1 as set forth in SEQ ID NO: 5, a heavy chain CDR2 as set forth in SEQ ID NO: 6, and a heavy chain CDR3 as set forth in SEQ ID NO: 7, or a heavy chain CDR1 as set forth in SEQ ID NO: 13, a heavy chain CDR2 as set forth in SEQ ID NO: 14, and a heavy chain CDR3 as set forth in SEQ ID NO: 15, and wherein the light chain variable region comprises a light chain CDR1 as set forth in SEQ ID NO: 8, a light chain CDR2 as set forth in SEQ ID NO: 9, and a light chain CDR3 as set forth in SEQ ID NO: 10, or a light chain CDR1 as set forth in SEQ ID NO: 16, a light chain CDR2 as set forth in SEQ ID NO: 17, and a light chain CDR3 as set forth in SEQ ID NO: 18.

32. The method of claim 31, wherein the binding molecule comprises a heavy chain variable region and a light chain variable region,
wherein the heavy chain variable region comprises a heavy chain CDR1 having the amino acid sequence as set forth in SEQ ID NO: 5, a heavy chain CDR2 having the amino acid sequence as set forth in SEQ ID NO: 6, and a heavy chain CDR3 having the amino acid sequence as set forth in SEQ ID NO: 7, and
wherein the light chain variable region comprises a light chain CDR1 having the amino acid sequence as set forth in SEQ ID NO: 8, a light chain CDR2 having the amino acid sequence as set forth in SEQ ID NO: 9, and a light chain CDR3 having the amino acid sequence as set forth in SEQ ID NO: 10.

33. The method of claim 31, wherein the binding molecule comprises a heavy chain variable region and a light chain variable region,
wherein the heavy chain variable region comprises a heavy chain CDR1 having the amino acid sequence as set forth in SEQ ID NO: 13, a heavy chain CDR2 having the amino acid sequence as set forth in SEQ ID NO: 14, and a heavy chain CDR3 having the amino acid sequence as set forth in SEQ ID NO: 15, and
wherein the light chain variable region comprises a light chain CDR1 having the amino acid sequence as set forth in SEQ ID NO: 16, a light chain CDR2 having the amino acid sequence as set forth in SEQ ID NO: 17, and a light chain CDR3 having the amino acid sequence as set forth in SEQ ID NO: 18.

34. The method of claim 27, wherein the binding molecule comprises a heavy chain variable region and a light chain variable region, wherein the heavy chain variable region comprises the amino acid sequence as set forth in SEQ ID NO: 11 or 19, and wherein the light chain variable region comprises the amino acid sequence as set forth in SEQ ID NO: 12 or 20.

35. The method of claim 34, wherein the heavy chain variable region comprises the amino acid sequence as set forth in SEQ ID NO: 11, and wherein the light chain variable region comprises the amino acid sequence as set forth in SEQ ID NO: 12.

36. The method of claim 34, wherein the heavy chain variable region comprises the amino acid sequence as set forth in SEQ ID NO: 19, and wherein the light chain variable region comprises the amino acid sequence as set forth in SEQ ID NO: 20.

37. The method of claim 27, wherein the binding molecule comprises an Fc region or a constant region.

38. The method of claim 37, wherein the Fc region is an Fc region of an IgA, IgD, IgE, IgM, IgG1, IgG2, IgG3, or IgG4 antibody, or a hybrid Fc region.

39. The method of claim 27, wherein the binding molecule comprises a heavy chain constant region and a light chain constant region, wherein the heavy chain constant region comprises the amino acid sequence as set forth in SEQ ID NO: 21, 23, 24, 26, 27, 28, or 29, and wherein the light chain constant region comprises the amino acid sequence as set forth in SEQ ID NO: 22 or 25.

40. The method of claim 39, wherein the heavy chain constant region comprises the amino acid sequence as set forth in SEQ ID NO: 21, and wherein the light chain constant region comprises the amino acid sequence as set forth in SEQ ID NO: 22.

41. The method of claim 39, wherein the heavy chain constant region comprises the amino acid sequence as set forth in SEQ ID NO: 23, 24, 26, 27, or 28, and wherein the light chain constant region comprises the amino acid sequence as set forth in SEQ ID NO: 25.

42. The method of claim 39, wherein the heavy chain constant region comprises the amino acid sequence as set forth in SEQ ID NO: 29.

43. The method of claim 27, wherein the binding molecule is selected from the group consisting of:
a binding molecule comprising a heavy chain as set forth in SEQ ID NO: 30 and a light chain as set forth in SEQ ID NO: 31;
a binding molecule comprising a heavy chain as set forth in SEQ ID NO: 32 and a light chain as set forth in SEQ ID NO: 33; and
a binding molecule comprising a heavy chain as set forth in SEQ ID NO: 34 and a light chain as set forth in SEQ ID NO: 35.

44. The method of claim 27, wherein the binding molecule is an antibody or a fragment thereof, wherein the antibody is a chimeric antibody, a humanized antibody, a bivalent, bispecific molecule, a minibody, a domain antibody, a bispecific antibody, an antibody mimetic, a unibody, a diabody, a triabody, or a tetrabody.

45. The method of claim 27, wherein the nervous system disease is a neurodegenerative disease or neuroinflammatory disease selected from the group consisting of stroke, dementia, Alzheimer's disease, Parkinson's disease, Huntington's disease, Niemann-Pick disease, multiple sclerosis, prion disease, Creutzfeldt-Jakob disease, frontotemporal dementia, dementia with Lewy bodies, amyotrophic lateral sclerosis, paraneoplastic syndrome, cortical degeneration syndrome, multiple system atrophy, progressive supranuclear palsy, nervous system autoimmune disease, spinocerebellar ataxia, inflammatory and neuropathic pain, cerebrovascular disease, spinal cord injury, and tauopathy.

46. A method for preventing or treating a nervous system disease in a subject in need thereof, the method comprising administering to the subject a pharmaceutically effective amount of an antibody-drug conjugate, wherein the antibody-drug conjugate comprises a heavy chain variable region and a light chain variable region,
wherein the heavy chain variable region comprises a heavy chain CDR1 having the amino acid sequence as set forth in SEQ ID NO: 5 or 13, a heavy chain CDR2 having the amino acid sequence as set forth in SEQ ID NO: 6 or 14, and a heavy chain CDR3 having the amino acid sequence as set forth in SEQ ID NO: 7 or 15, and
wherein the light chain variable region comprises a light chain CDR1 having the amino acid sequence as set forth in SEQ ID NO: 8 or 16, a light chain CDR2 having the amino acid sequence as set forth in SEQ ID NO: 9 or 17, and a light chain CDR3 having the amino acid sequence as set forth in SEQ ID NO: 10 or 18.
